(19) United States
(12) Patent Application Publication
(10) Pub. No.: US 2004/0267102 A1
(43) Pub. Date: Dec. 30, 2004

(54) Diagnostic feature extraction in dermatological examination

(76) Inventors: Victor Nickolaevick Skladnev, Vaucluse (AU); Alexander Gutenev, North Narrabeen (AU); Scott Menzies, Sydney (AU); Leanne Margaret Bischof, Putney (AU); Gustave Francois Talbot, North Ryde (AU); Edmond Joseph Breen, Berowra Heights (AU); Michael James Buckley, Pennant Hills (AU)

Correspondence Address: GOTTLIEB RACKMAN & REISMAN PC, 270 MADISON AVENUE, 8TH FLOOR, NEW YORK, NY, 100160601

(21) Appl. No.: 10/478,078

(22) PCT Filed: May 17, 2002
(86) PCT No.: PCT/AU02/00604

(30) Foreign Application Priority Data
May 18, 2001 (AU) PR 5096

(51) Int. Cl. G06K 9/00; A61B 5/00

(52) U.S. Cl. 600/315; 382/128

(57) ABSTRACT

An automated dermatological examination system captures an image including a lesion. The image is divided into lesion and non-lesion areas, and the lesion area is analysed to quantify features for use in diagnosis of the lesion. Features of lesion colour are extracted (step 502) and the shape of the lesion is analysed (step 504). Features of lesion texture and symmetry are derived (step 506) as are measures of categorical symmetry within the lesion (step 508).

### FIELD OF THE INVENTION

The present invention relates to the examination of dermatological anomalies and, in particular, to the automatic extraction of features relating to the colour, shape, texture and symmetry of skin lesions and like structures.

### BACKGROUND

Malignant melanoma is a form of cancer due to the uncontrolled growth of melanocytic cells under the surface of the skin. These pigmented cells are responsible for the brown colour in skin and freckles. Malignant melanoma is one of the most aggressive forms of cancer. The interval between a melanoma site becoming malignant or active and the probable death of the patient in the absence of treatment may be short, of the order of only six months. Deaths occur due to the spread of the malignant melanoma cells beyond the original site through the blood stream and into other parts of the body. Early diagnosis and treatment is essential for favourable prognosis.

However, the majority of medical practitioners are not experts in the area of dermatology and each might see only a few melanoma lesions in any one year. As a consequence, the ordinary medical practitioner has difficulty in assessing a lesion properly.

The examination of skin lesions and the identification of skin cancers such as melanoma have traditionally been performed with the naked eye. More recently, dermatologists have used hand-held optical magnification devices generally known as a dermatoscope (or Episcope). Such devices typically incorporate a source of light to illuminate the area under examination and a flat glass window which is pressed against the skin in order to flatten the skin and maximise the area of focus. The physician looks through the instrument to observe a magnified and illuminated image of lesion. An expert dermatologist can identify over 70 different morphological characteristics of a pigmented lesion. The dermatoscope is typically used with an index matching medium, such as mineral oil which is placed between the window and the patient's skin. The purpose of the “index matching oil” is to eliminate reflected light due to a mis-match in refractive index between skin and air. Whilst the dermatoscope provides for a more accurate image to be represented to the physician, the assessment of the lesion still relies upon the manual examination and the knowledge and experience of the physician.

More recently automated analysis arrangements have been proposed which make use of imaging techniques to provide an assessment of the lesion and a likelihood as to whether or not the lesion may be cancerous. Such arrangements make use of various measures and assessments of the nature of the lesion to provide the assessment as to whether or not it is malignant. Such measures and assessments can include shape analysis, colour analysis and texture analysis, amongst others,

A significant problem of such arrangements is the computer processing complexity involved in performing imaging processes and the need or desire for those processes to be able to be performed as quickly as possible. If processing can be shortened, arrangements may be developed whereby an assessment of a lesion can be readily provided to the patient, possibly substantially coincident with optical examination by the physician and/or automated arrangement (ie. a “real-time” diagnosis).

### SUMMARY OF THE INVENTION

The invention relates to the automatic examination of an image including a lesion. The image is segmented into lesion and non-lesion areas and features of the lesion area are automatically extracted to assist in diagnosis of the lesion. The extracted features include features of lesion colour, shape, texture and symmetry.

It is an object of the present invention to substantially overcome, or at least ameliorate, one or more deficiencies of prior art arrangements.

According to a first aspect of the invention there is provided a method of quantifying features of a skin lesion for use in diagnosis of the skin lesion, the method comprising the steps of:

obtaining an image of an area of skin including the lesion;

segmenting the image into a lesion area and a non-lesion area;

quantifying at least one colour feature of the lesion area;

quantifying at least one shape feature of the lesion area;

calculating at least one symmetry measure descriptive of the distribution of classified regions within the lesion area; and

storing the at least one colour feature, the at least one shape feature and the at least one symmetry measure for use in diagnosis of the skin lesion.

According to a second aspect of the invention there is provided a method of quantifying features of a skin lesion for use in diagnosis of the skin lesion, the method comprising the steps of:

obtaining a calibrated colour image of an area of skin including the lesion, the image comprising a set of visual elements;

segmenting the image into a lesion area and a non-lesion area;

allocating each visual element in the lesion area to a corresponding one of a predefined set of colour classes;

calculating at least one statistic describing the distribution of the allocated visual elements; and

storing the at least one statistic for further processing as a feature of the lesion.

According to a further aspect of the invention there is provided a method of quantifying features of a skin lesion for use in diagnosis of the skin lesion, the method comprising the steps of:

obtaining a calibrated image of an area of skin including the lesion, the image comprising a set of visual elements;

segmenting the image into a lesion area and a non-lesion area;

assigning a constant value to each visual element in the lesion area to form a binary lesion image;

isolating one or more notches in the binary lesion image;

calculating at least one statistic describing the one or more notches; and

storing the at least one statistic for further processing as a feature of the lesion.

According to a further aspect of the invention there is provided a method of quantifying features of a skin lesion for use in diagnosis of the skin lesion, the method comprising the steps of:

obtaining a calibrated colour image of an area of skin including the lesion, the image comprising a set of visual elements;

dividing the image into a lesion area and a non-lesion area;

segmenting the lesion area into one or more classes, each class comprising at least one sub-region, such that all visual elements in a class satisfy a predefined criterion;

calculating at least one statistic describing the spatial distribution of the classes;

storing the at least one statistic for her processing as a feature of the lesion.

According to a further aspect of the invention there is provided a method of quantifying features of a skin lesion for use in diagnosis of the skin lesion, the method comprising the steps of: obtaining a calibrated colour image of an area of skin including the lesion, the image comprising a set of visual elements described by coordinates in a colour space;

segmenting the image into a lesion area and a non-lesion area;

comparing, for each visual element, the coordinates with a predefined lookup table;

allocating the visual element to a corresponding one of a predefined set of colours based on said comparison;

calculating at least one statistic describing the distribution of the allocated visual elements; and

storing the at least one statistic for further processing as a feature of the lesion.

According to a further aspect of the invention there is provided a method of quantifying features of a skin lesion for use in diagnosis of the skin lesion, the method comprising the steps of:

obtaining a calibrated image of an area of skin including the lesion, the image comprising a set of visual elements;

segmenting the image into a lesion area and a non-lesion area;

assigning a constant value to each visual element in the lesion area to form a binary lesion image;

performing a morphological closing of the binary lesion image to form a closed lesion image;

subtracting the binary lesion image from the closed lesion image to produce one or more difference regions;

performing a morphological opening of the one or more difference regions to produce one or more notches;

calculating at least one statistic describing the one or more notches; and

storing the at least one statistic for further processing as a feature of the lesion

According to a further aspect of the invention there is provided a method of quantifying features of a skin lesion for use in diagnosis of the skin lesion, the method comprising the steps of:

obtaining a calibrated image of an area of skin including the lesion, the image comprising a set of visual elements wherein each visual element has a value;

calculating a lesion boundary that segments the image into a lesion area and a non-lesion area;

calculating an average of the value of each visual element lying on the lesion boundary to form a boundary average;

generating a plurality of outer contours such that each outer contour follows the lesion boundary at a predetermined distance;

generating a plurality of inner contours such that each inner contour follows the lesion boundary at a predetermined distance;

for each one of the inner and outer contours, calculating an average of the value of each visual element lying on the contour to form a contour average;

plotting the contour averages and boundary average against distance to form an edge profile;

normalising the edge profile;

finding a mid-point of the normalised edge profile,

defining a left shoulder region lying within a predefined distance range of the mid-point,

defining a right shoulder region lying within the predefined distance range;

calculating a right area from the right shoulder region and a left area from the left shoulder area;

calculating an edge abruptness measure as the sum of the left area and the right area; and

storing the edge abruptness measure for further processing as a feature of the lesion.

According to a further aspect of the invention there is provided a method of quantifying features of a skin lesion for use in diagnosis of the skin lesion, the method comprising the steps of:

obtaining a calibrated colour image of an area of skin including the lesion, the image comprising a set of visual elements;

dividing the image into a lesion area and a non-lesion area;

associating each visual element in the lesion with a corresponding one of a predefined set of colour classes;

segmenting the lesion area into one or more classes, each class having at least one-sub-region, such that all visual elements in a class are associated with a common one of the colour classes; and

calculating at least one statistic describing the spatial distribution of the classes;

storing the at least one statistic for further processing as a feature of the lesion.

According to a further aspect of the invention there is provided a method of quantifying features of a skin lesion for use in diagnosis of the skin lesion, the method comprising the steps of:

obtaining a calibrated colour image of an area of skin including the lesion, the image comprising a set of visual elements having a descriptive parameter;

dividing the image into a lesion area and a non-lesion area;

constructing a cumulative histogram of all visual elements in the lesion area according to the descriptive parameter; dividing the cumulative histogram into a plurality of sectors;

segmenting the lesion area into one or more classes, each class having at least one sub-region, such that all visual elements in a class are associated with a common one of the plurality of sectors; and

calculating at least one statistic describing the spatial distribution of the classes;

storing the at least one statistic for further processing as a feature of the lesion.

According to a further aspect of the invention there is provided a method of quantifying features of a skin lesion for use ill diagnosis of the skin lesion, the method comprising the steps of:

obtaining a calibrated colour image of an area of skin including the lesion, the image comprising a set of visual elements defined in a first colour space;

dividing the image into a lesion area and a non-lesion area;

transforming the first colour space to a two-dimensional colour space using a predetermined transform;

forming a bivariate histogram of the visual elements in the lesion area;

identifying one or more seed regions based on the peaks of the bivariate histogram;

dividing a populated part of the two-dimensional colour space into a plurality of category regions derived from the seed regions;

segmenting the lesion area into one or more classes, each class comprising at least one sub-region, such that all visual elements in a class are associated with a common one of the category regions;

calculating at least one statistic describing the spatial distribution of the classes; and

storing the at least one statistic for farther processing as a feature of the lesion.

According to another aspect of the invention, there is provided an apparatus for implementing any one of the aforementioned methods.

Other aspects of the invention are also disclosed.

### BRIEF DESCRIPTION OF THE DRAWINGS

At least one embodiment of the present invention will now be described with reference to the drawings, in which:

FIG. 1 is a schematic block diagram representation of a computerised dermatological examination system;

FIG. 2 is a schematic representation of the camera assembly of FIG. 1 when in use to capture an image of a lesion;

FIG. 3 is a schematic block diagram representation of a data flow of the system of FIG. 1;

FIG. 4 is a flow diagram of the imaging processes of FIG. 3;

FIG. 5 is a flow diagram of the feature detection system of FIG. 4;

FIG. 6A is a flow diagram showing the separation of a lesion image into colour classes;

FIG. 6B is a flow diagram of a method of generating a look-up table as used in the method of FIG. 6A;

FIG. 6C is a flow diagram of a method of defining a Blue-White Veil (BWV) region in RGB space;

FIG. 6D is a flow diagram of the identification of BWV colour in a lesion image;

FIG. 7A shows a single plane of a histogram in RGB space;

FIG. 7B shows the histogram of FIG. 7A separated into blue and brown classes;

FIG. 7C shows the histogram of FIG. 7A further subdivided into different brown classes;

FIG. 8A is a flow diagram of the extraction of shape features of a lesion image;

FIG. 8B is a flow diagram showing the notch analysis process of FIG. 8A;

FIG. 8C is a flow diagram showing the calculation of the fractal dimension measure of FIG. 8A;

FIG. 8D is a flow diagram showing the calculation of an edge abruptness measure of a lesion image;

FIG. 9A shows an example of a lesion mask;

FIG. 9B shows a best-fit ellipse fitted to the lesion mask of FIG. 9A;

FIG. 9C illustrates the notch analysis method of FIG. 8B;

FIG. 9D shows geodesic contours within a notch of FIG. 9C;

FIG. 9E shows the boundary contours of FIG. 8D;

FIG. 9F shows an edge profile of the lesion of FIG. 9E;

FIG. 10A shows a flow diagram of the extraction of features relating to texture and symmetry of a lesion;

FIG. 10B is a flow diagram of the comparison between binary and grey-scale measures of FIG. 10A;

FIG. 10C is a flow diagram of the “data flip” measures of FIG. 10A;

FIG. 10D is a flow diagram of the radial measure extraction of FIG. 10A;

FIG. 11A shows a binary lesion mask with a best-fit ellipse superimposed;

FIG. 11B shows a grey-level lesion mask with a best-fit ellipse superimposed;

FIG. 11C shows a comparison of the best-fit ellipses of FIG. 11A and FIG. 11B;

FIG. 11D illustrates the image flip measures of FIG. 10C;

FIGS. 12A-H illustrate the radial feature extraction of FIG. 10D;

FIG. 13A is a flow diagram of categorical symmetry measures;

FIG. 13B is a flow chart of the segmentation process of FIG. 13A based on absolute colour;

FIG. 13C is a flow chart of the segmentation process of FIG. 13A based on one-dimensional histograms;

FIG. 13D is a flow chart of the segmentation process of FIG. 13A based on relative colour segmentation;

FIG. 13E is a flow chart showing detail of the formation of the bivariate histogram of FIG. 13D;

FIG. 13F is a flow chart showing more detail of the identification of the seeds in the process of FIG. 13D;

FIG. 14A illustrates the categorical symmetry measures of FIG. 13A;

FIG. 14B illustrates the segmentation process of FIG. 13C;

FIG. 14C is a view of a lesion in terms of variable PC1;

FIG. 14D is a view of the lesion shown in FIG. 14C, in terms of variable PC2;

FIG. 14E shows a bivariate histogram of lesion data in a space defined by variables PC1 and PC2;

FIG. 14F shows a set of candidate peaks derived from the histogram of FIG. 14E;

FIG. 14G shows the candidate peaks of FIG. 14F after a merging operation;

FIG. 14H shows the seed objects of FIG. 14G with labelling applied;

FIG. 14I shows the labels of FIG. 14H as applied to the candidate seeds of FIG. 14F;

FIG. 14J shows the histograms space defined by variables PC1 and PC2 segmented according to the categories of FIG. 14I;

FIG. 14K shows the segmentation of FIG. 14J restricted to the populated part of the histogram of FIG. 14B;

FIG. 14L shows the lesion from FIGS. 14C and 14D segmented in accordance with the categories of FIG. 14K;

FIG. 15 is a schematic block diagram of a computer system upon which the processing described can be practiced.

### DETAILED DESCRIPTION

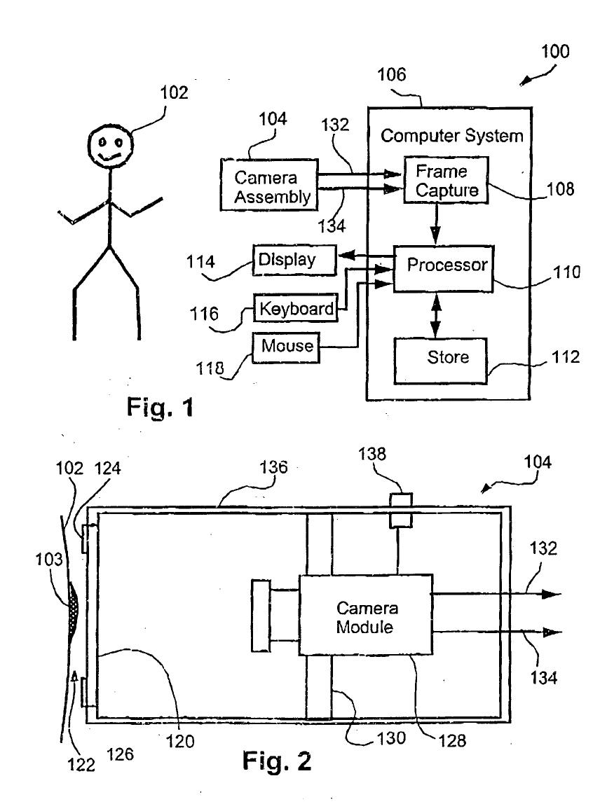

FIG. 1 shows an automated dermatological examination system 100 in which a camera assembly 104 is directed at a portion of a patient 102 in order to capture an image of the skin of the patient 102 and for which dermatological examination is desired. The camera assembly 104 couples to a computer system 106 which incorporates a frame capture board 108 configured to capture a digital representation of the image formed by the camera assembly 104. The frame capture board 108 couples to a processor 110 which can operate to store the captured image in a memory store 112 and also to form various image processing activities on the stored image and variations thereof that may be formed from such processing and/or stored in the memory store 112. Also coupled to the computer system via the processor 110 is a display 114 by which images captured and/or generated by the system 106 may be represented to the user or physician, as well as keyboard 116 and mouse pointer device 118 by which user commands may be input

As seen in FIG. 2, the camera assembly 104 includes a chassis 136 incorporating a viewing window 120 which is placed over the region of interest of the patient 102 which, in this case, is seen to incorporate a lesion 103. The window 120 incorporates on an exterior surface thereof and arranged in the periphery of the window 120 a number of colour calibration portions 124 and 126 which can be used as standardised colours to provide for colour calibration of the system 100. Such ensures consistency between captured images and classification data that may be used in diagnostic examination by the system 100. As with the dermatoscope as described above, an index matching medium such as oil is preferably used in a region 122 between the window 120 and the patient 102 to provide the functions described above.

The camera assembly 104 further includes a camera module 128 mounted within the chassis and depending from supports 130 in such a manner that the camera module 128 is fixed in its focal length upon the exterior surface of tile glass window 120, upon which the patient's skin is pressed. In this fashion, the optical parameters and settings of tile camera module 128 may be preset and need not be altered for the capture of individual images. The camera module 128 includes an image data output 132 together with a data capture control signal 134, for example actuated by a user operable switch 138. The control signal 134 may be used to actuate the frame capture board 108 to capture the particular frame image currently being output on the image connection 132. As a consequence, the physician, using the system 100, has the capacity to move the camera assembly 104 about the patient and into an appropriate position over the lesion 103 and when satisfied with the position (as represented by a real-time image displayed on the display 114), may capture the particular image by depression of the switch 138 which actuates the control signal 134 to cause the frame capture board 108 to capture the image.

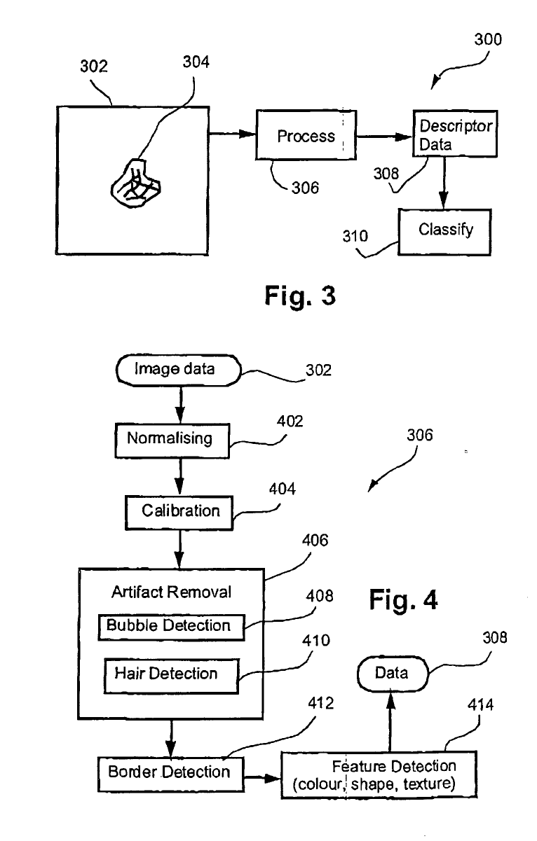

FIG. 3 depicts a generalised method for diagnosis using imaging that is performed by the system 100. An image 302, incorporating a representation 304 of the lesion 103, forms an input to the diagnostic method 300. The image 302 is manipulated by one or more processes 306 to derive descriptor data 308 regarding the nature of the lesion 103. Using the descriptor data 308, a classification 310 may be then performed to provide to the physician with information aiding a diagnosis of the lesion 103.

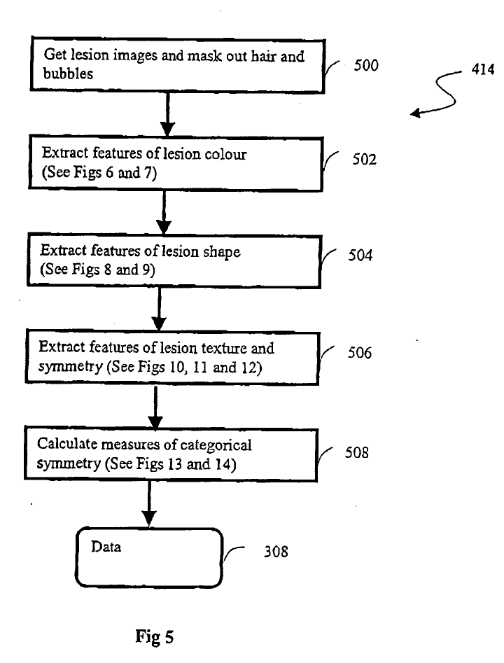

FIG. 4 shows a further flow chart representing the various processes formed within the process module 306. Initially, image data 302 is provided to a normalising process 402 which acts to compensate for light variations across the surface of the image. The normalised image is then provided to a calibration process 404 which operates to identify the calibration regions 124 and 126, and to note the colours thereof, so that automated calibration of those detected colours may be performed in relation to reference standards stored within the computer system 106. With such colours within the image 302 may be accurately identified in relation to those calibration standards.

The calibrated image is then subjected to artifact removal 406 which typically includes bubble detection 408 and hair detection 410. Bubble detection acts to detect the presence of bubbles in the index matching oil inserted into the space 122 and which can act to distort the image detected. Hair detection 410 operates to identify hair within the image and across the surface of the skin and so as to remove the hair from the image process. Bubble detection and hair detection processes are known in art and any one of a number of known arrangements may be utilised for the purposes of the present disclosure. Similarly, normalising and calibration processes are also known.

After artifacts are removed in step 406, border detection 412 is performed to identify the outline/periphery of the lesion 103. Border detection may be performed by manually tracing an outline of the lesion as presented on the display 114 using the mouse 118. Alternatively, automated methods such as region growing may be used and implemented by the computer system 106.

Once the border is detected, feature detection 414 is performed upon pixels within the detected border to identify features of colour, shape and texture, amongst others, those features representing the descriptor data 308 that is stored and is later used for classification purposes.

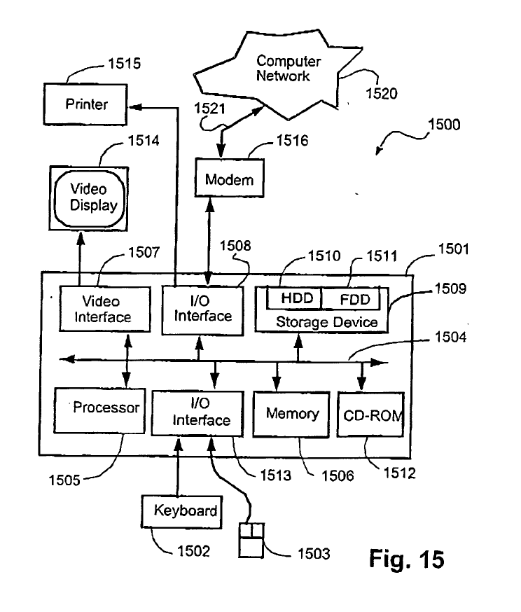

The methods described here, and generally depicted in FIG. 1, may be practiced using a general-purpose computer system 1500, such as that shown in FIG. 15 wherein the described processes of lesion feature extraction may be implemented as software, such as an application program executing within the computer system 1500. The computer system 1500 may substitute for the system 106 or may operate in addition thereto. In the former arrangement the system 1500 represents a detailed depiction of the components 110-118 of FIG. 1. In particular, the steps of the methods are effected by instructions in the software that are carried out by the computer. The software may be divided into two separate parts in which one part is configured for carrying out the feature extraction methods, and another part to manage the user interface between the latter and the user. The software may be stored in a computer readable medium, including the storage devices described below, for example. The software is loaded into the computer from the computer readable medium, and then executed by the computer. A computer readable medium having such software or computer program recorded on it is a computer program product. The use of the computer program product in the computer preferably effects an advantageous apparatus for dermatological processing.

The computer system 1500 comprises a computer module 1501, input devices such as a keyboard 1502 and mouse 1503, output devices including a printer 1515 and a display device 1514. A Modulator-Demodulator (Modem) transceiver device 1516 may be used by the computer module 1501 for communicating to and from a communications network 1520, for example connectable via a telephone line 1521 or other functional medium. The modem 1516 can be used to obtain access to the Internet, and other network systems, such as a Local Area Network (LAN) or a Wide Area Network (WAN).

The computer module 1501 typically includes at least one processor unit 1505, a memory unit 1506, for example formed from semiconductor random access memory (RAM) and read only memory (ROM), input/output (I/O) interfaces including a video interface 1507, and an I/O interface 1513 for the keyboard 1502 and mouse 1503 and optionally a joystick (not illustrated), and an interface 1508 for the modem 1516. A storage device 1509 is provided and typically includes a hard disk drive 1510 and a floppy disk drive 1511. A magnetic tape drive (not illustrated) may also be used. A CD-ROM drive 1512 is typically provided as a non-volatile source of data The components 1505 to 1513 of the computer module 1501, typically communicate via an interconnected bus 1504 and in a manner which results in a conventional mode of operation of the computer system 1500 known to those in the relevant art. Examples of computers on which the described arrangements can be practised include IBM-PC's and compatibles, Sun Sparcstations or alike computer systems.

Typically, the application program is resident on the hard disk drive 1510 and read and controlled in its execution by the processor 1505. Intermediate storage of the program and any data fetched from the network 1520 may be accomplished using the semiconductor memory 1506, possibly in concert with the hard disk drive 1510. In some instances, the application program may be supplied to the user encoded on a CD-ROM or floppy disk and read via the corresponding drive 1512 or 1511, or alternatively may be read by the user from the network 1520 via the modem device 1516. Still further, the software can also be loaded into the computer system 1500 from other computer readable media. The term “computer readable medium” as used herein refers to any storage or transmission medium that participates in providing instructions and/or data to the computer system 1500 for execution and/or processing. Examples of storage media include floppy disks, magnetic tape, CD-ROM, a hard disk drive, a ROM or integrated circuit, a magneto-optical disk, or a computer readable card such as a PCMCIA card and the like, whether or not such devices are internal or external of the computer module 1501. Examples of transmission media include radio or infra-red transmission channels as well as a network connection to another computer or networked device, and the Internet or Intranets including e-mail transmissions and information recorded on Websites and the like.

The processing methods may alternatively be implemented in dedicated hardware such as one or more integrated circuits performing the described functions or sub functions. Such dedicated hardware may include graphic processors, digital signal processors, or one or snore microprocessors and associated memories.

The general approach to lesion feature extraction according to the present disclosure is illustrated in FIG. 5. In step 500, the lesion image is retrieved from memory store 112 by the processor 110 for processing. The lesion image maybe regarded as a set of images, with each member of the set portraying the lesion in a different manner. For example, the lesion may be portrayed as a binary mask or a grey-level mask.

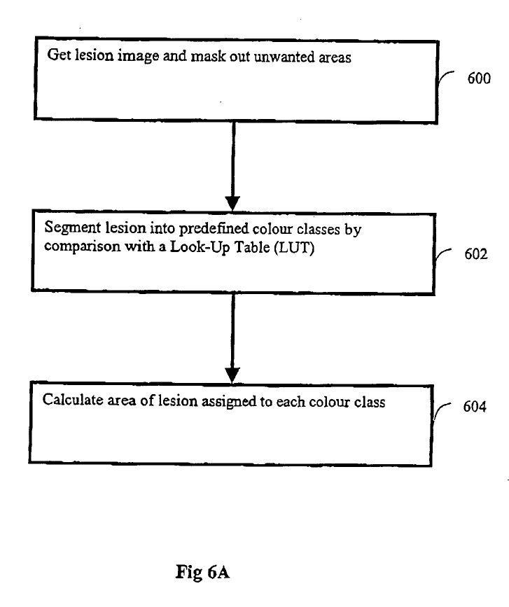

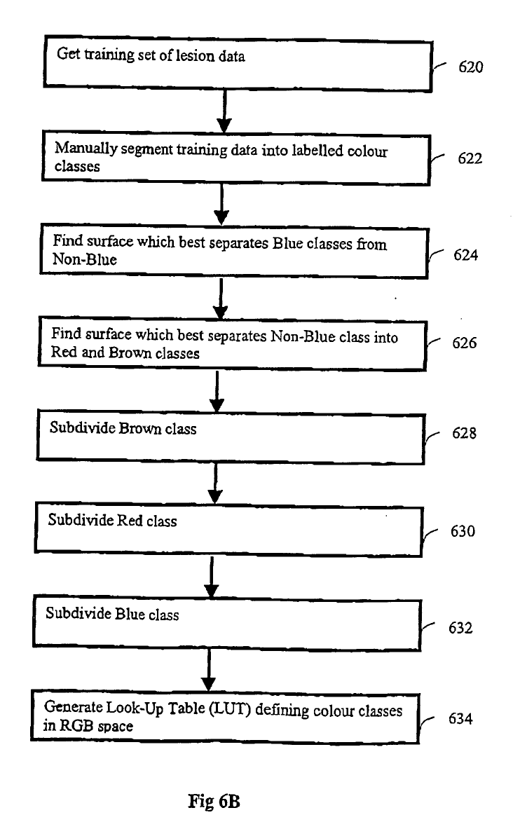

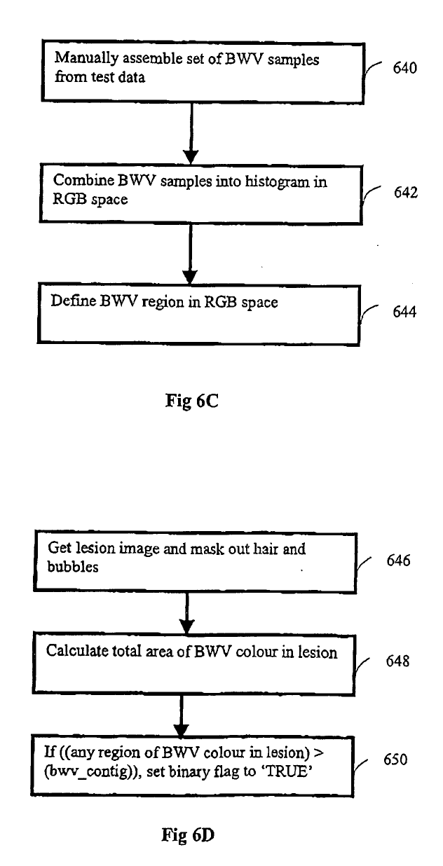

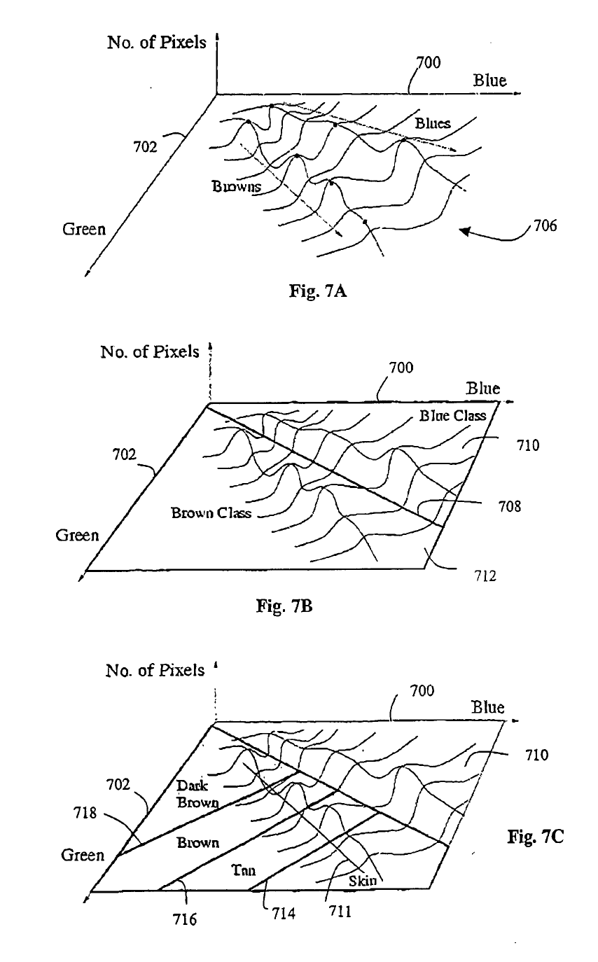

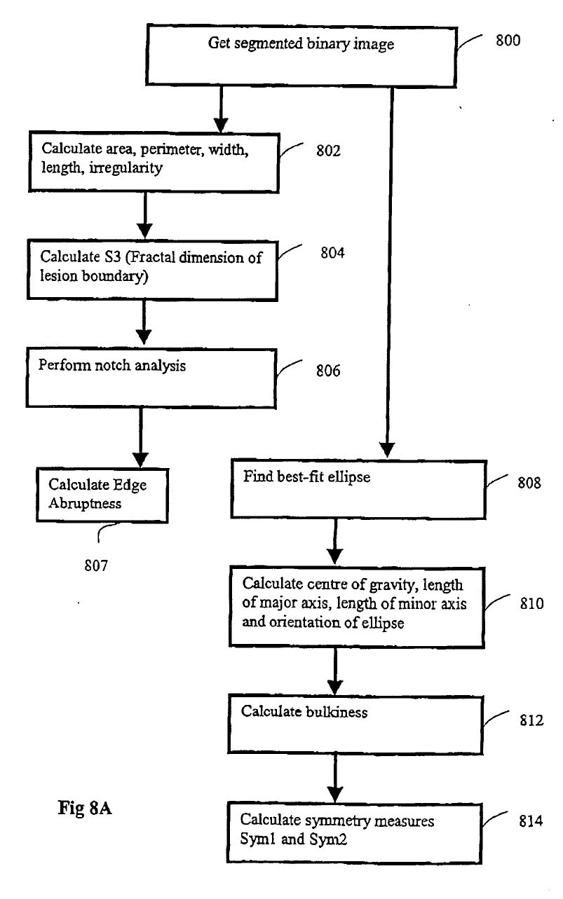

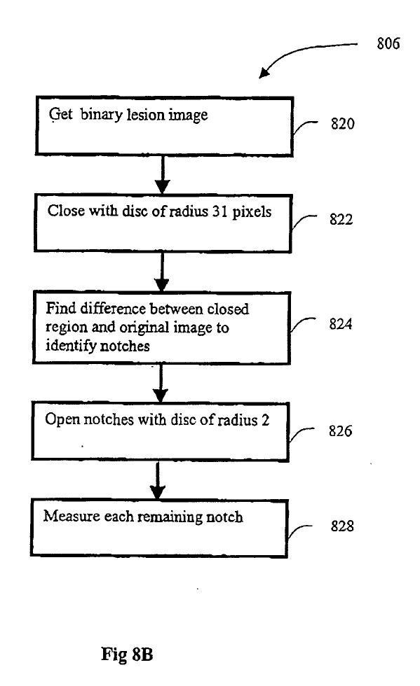

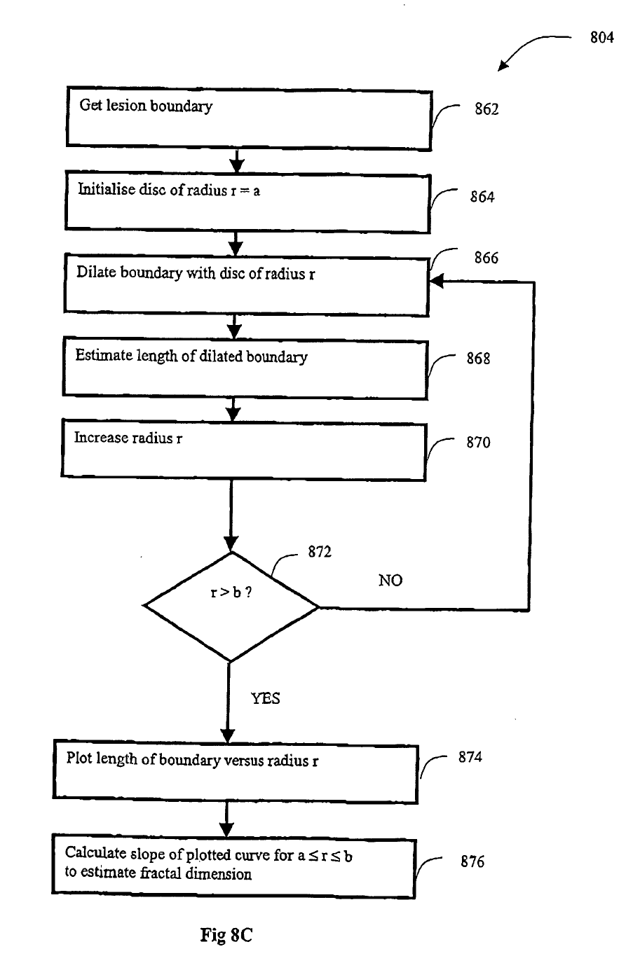

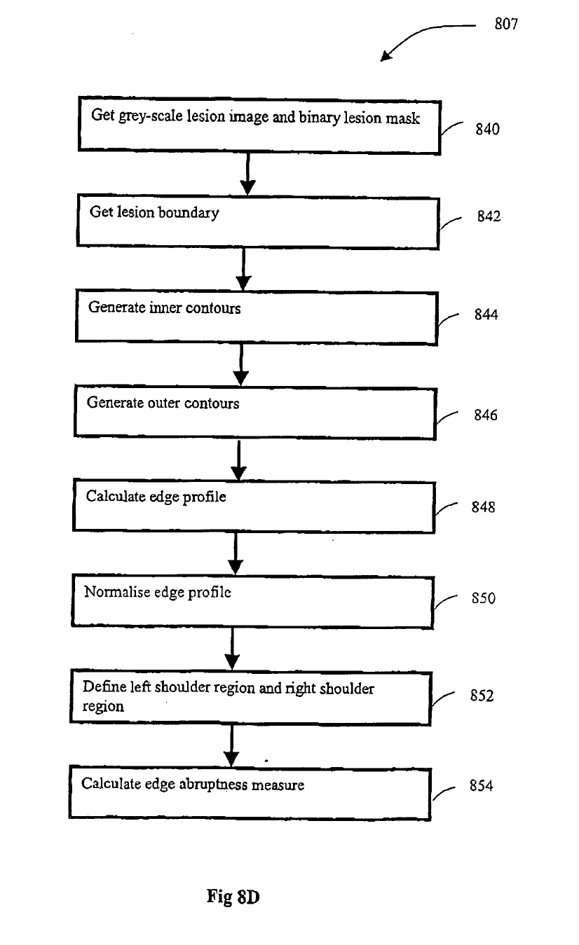

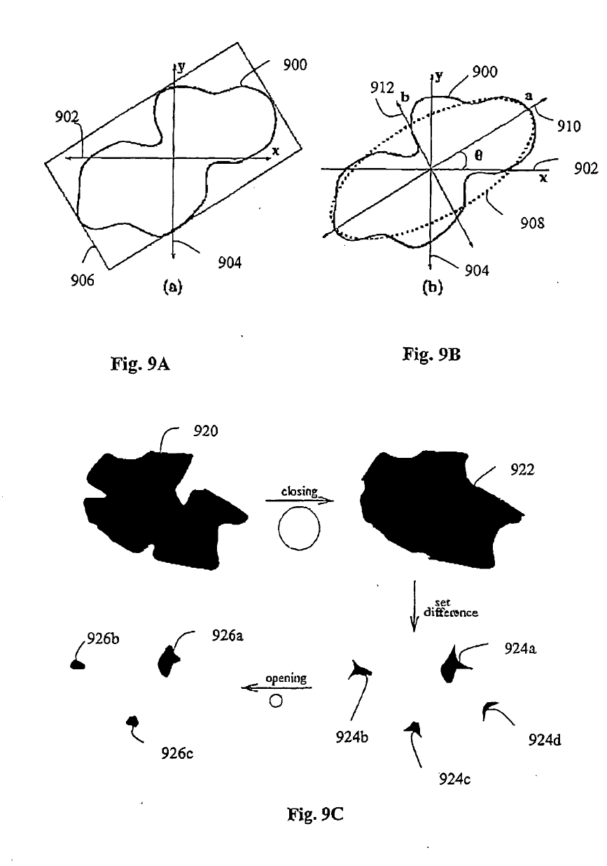

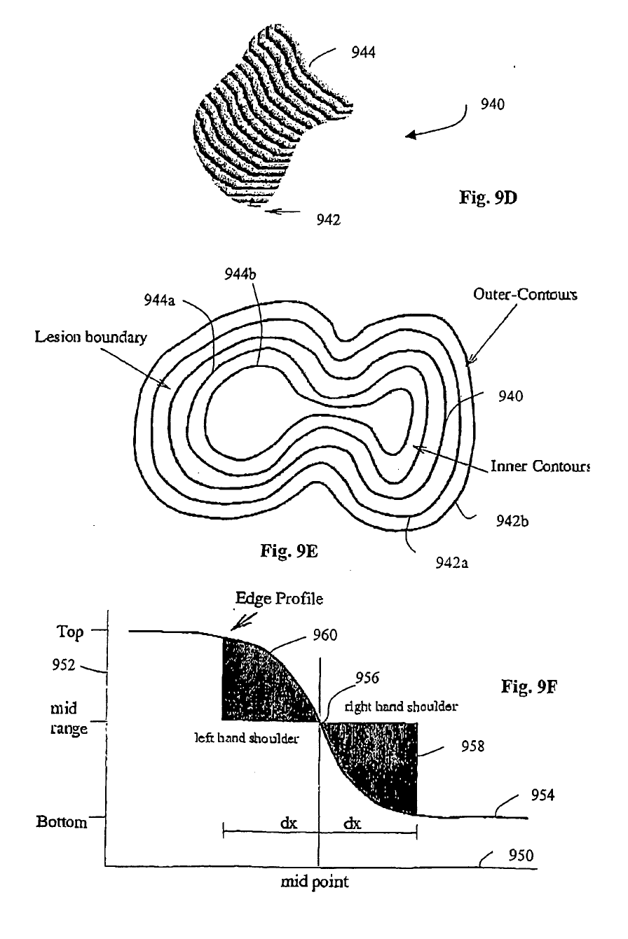

In step 502 features of lesion colour are extracted. These colour features will be described in more detail with reference to FIGS. 6 and 7. In step 504 features of lesion shape are extracted, as will be described in more detail with reference to FIGS. 8 and 9.

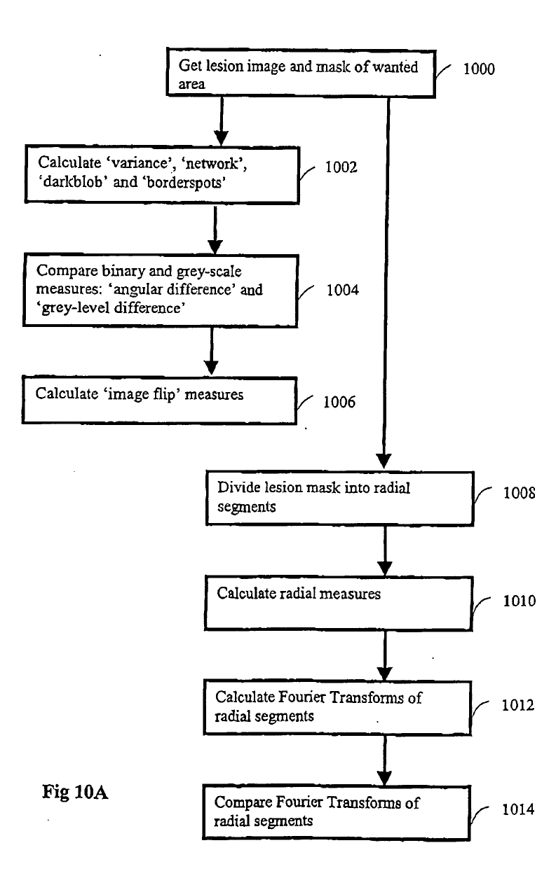

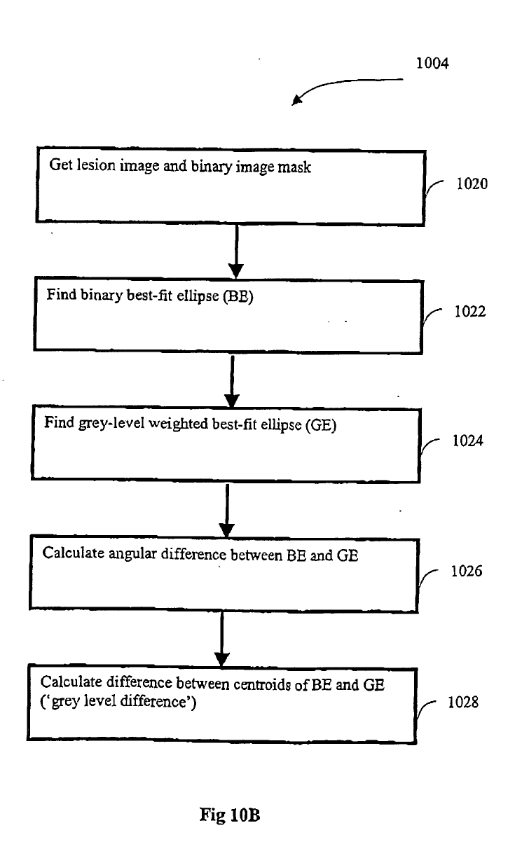

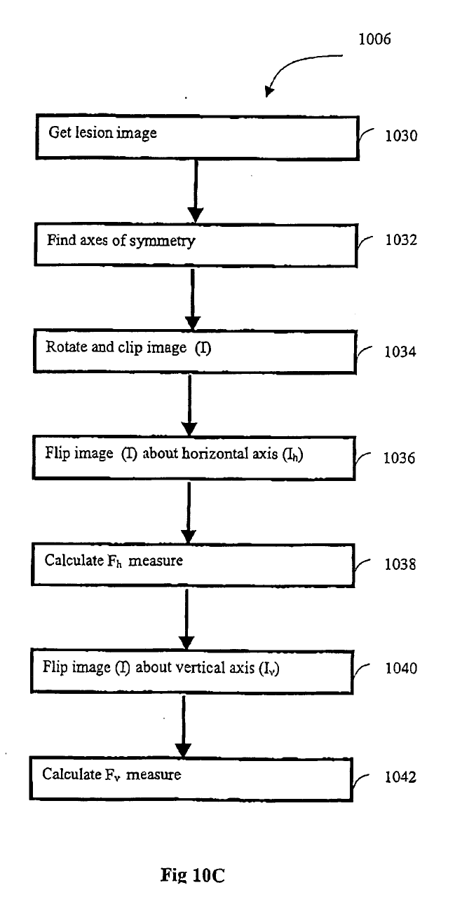

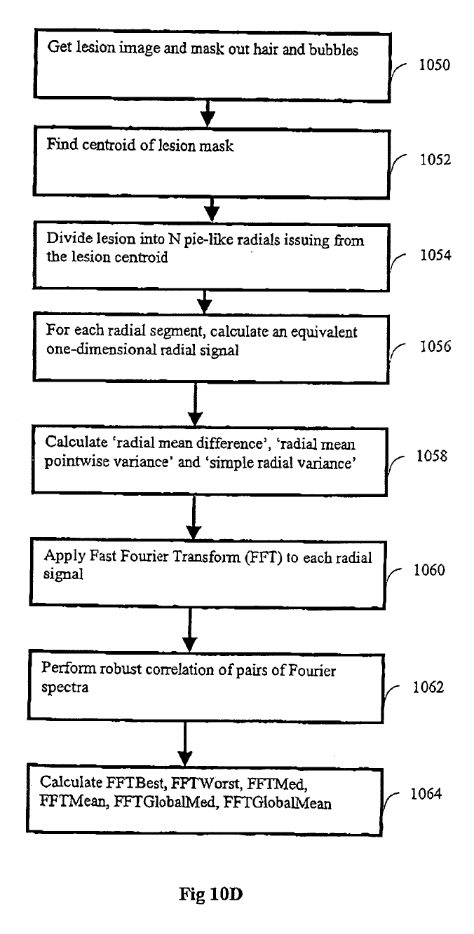

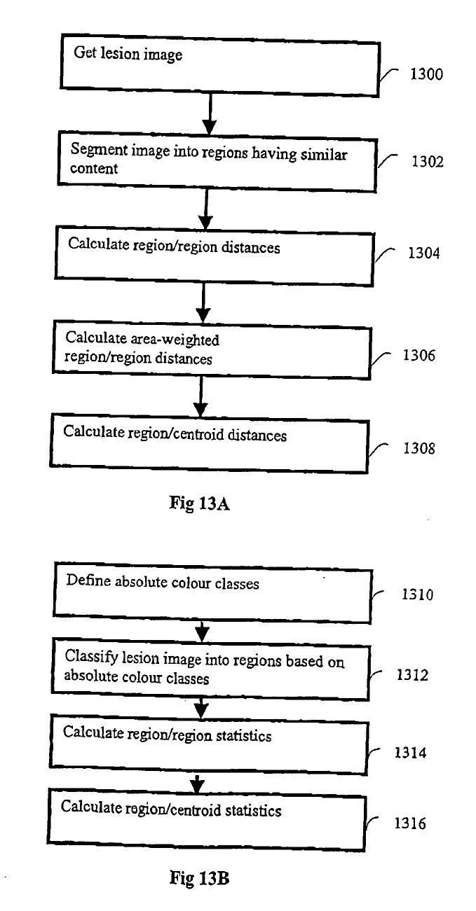

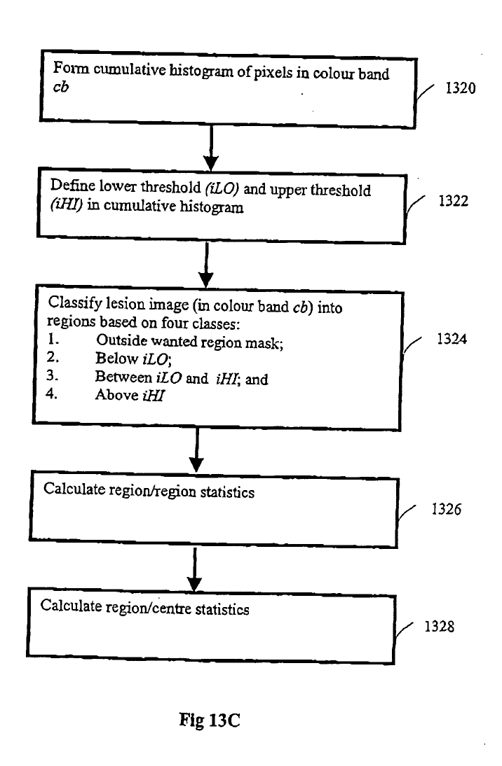

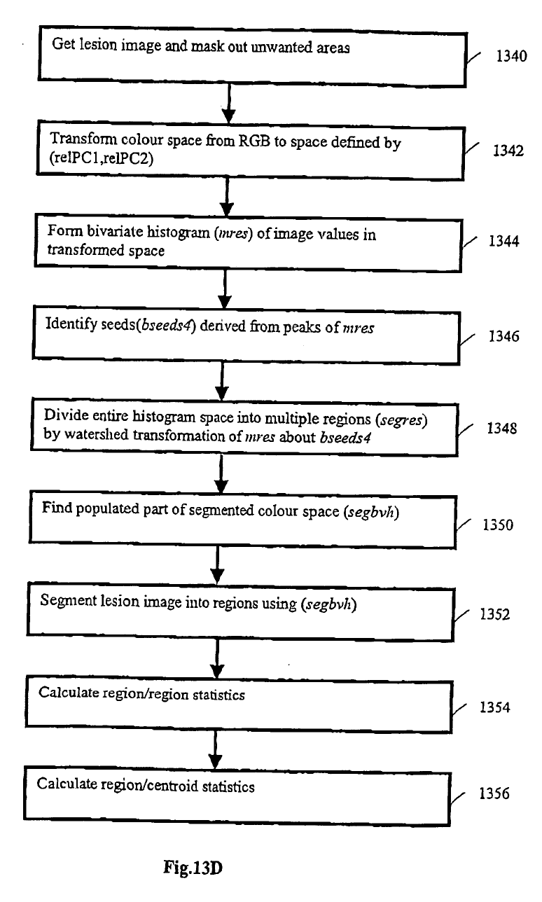

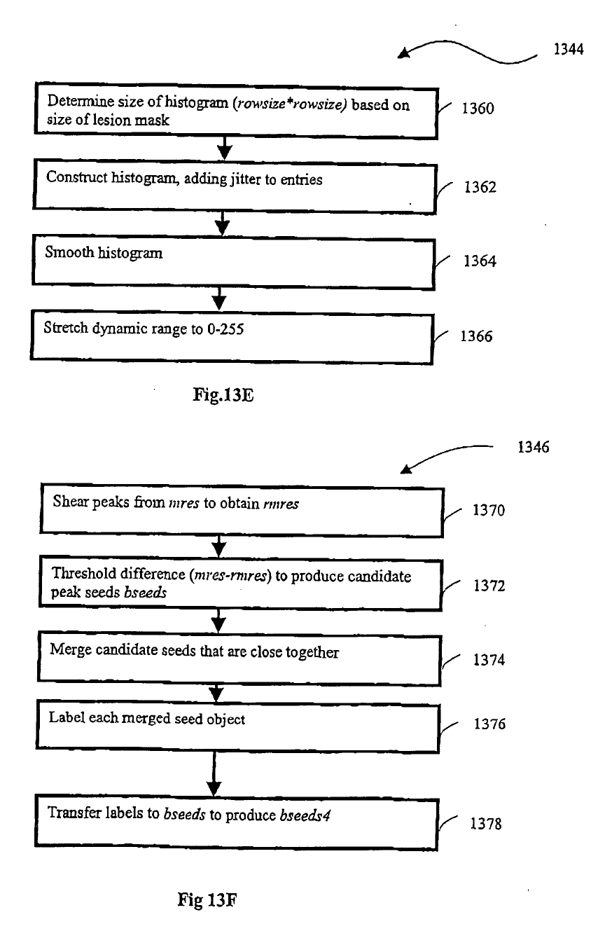

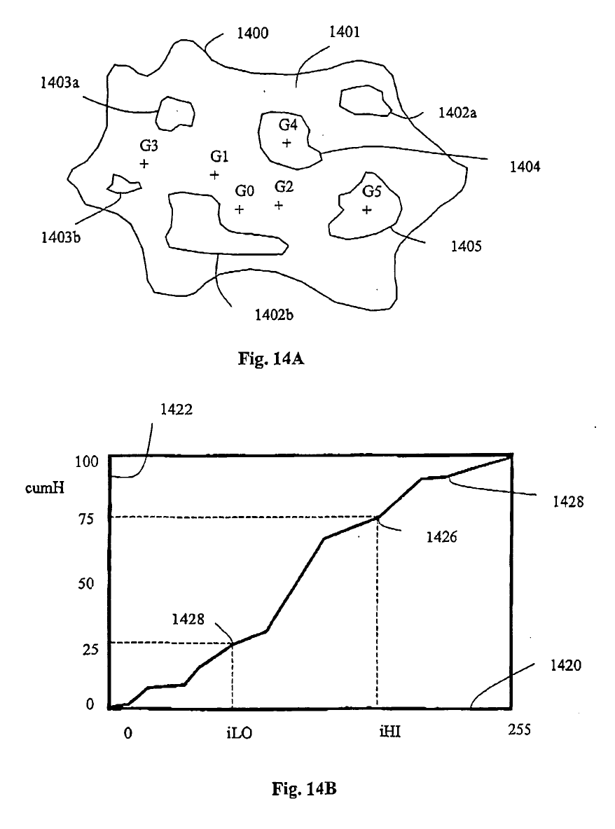

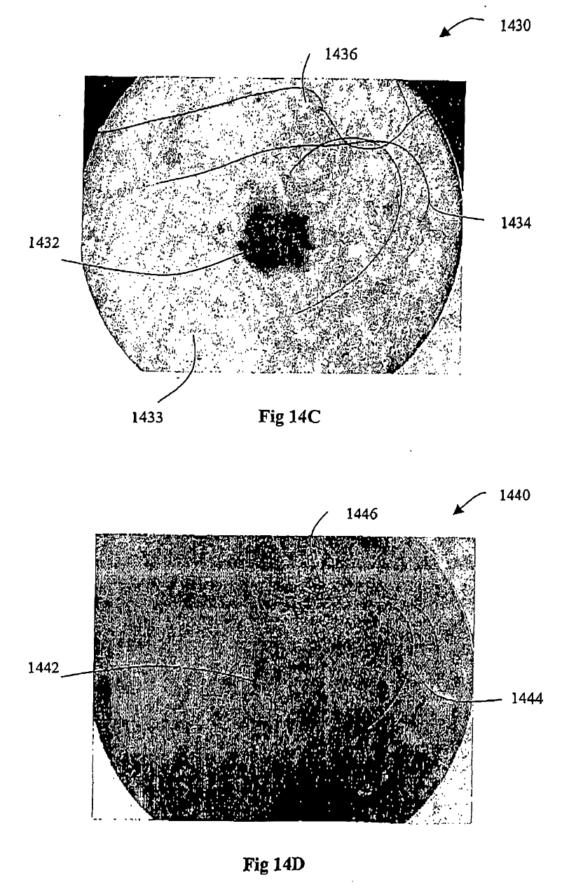

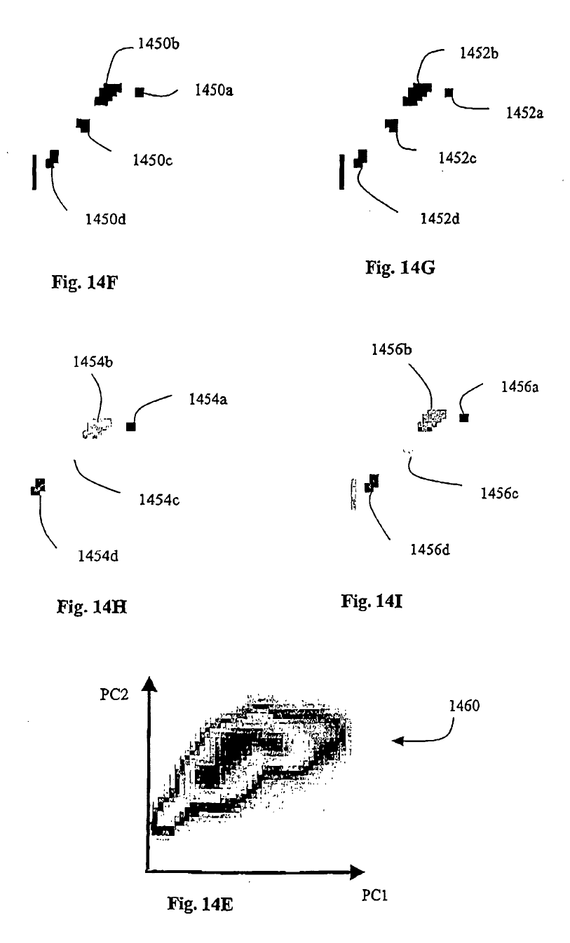

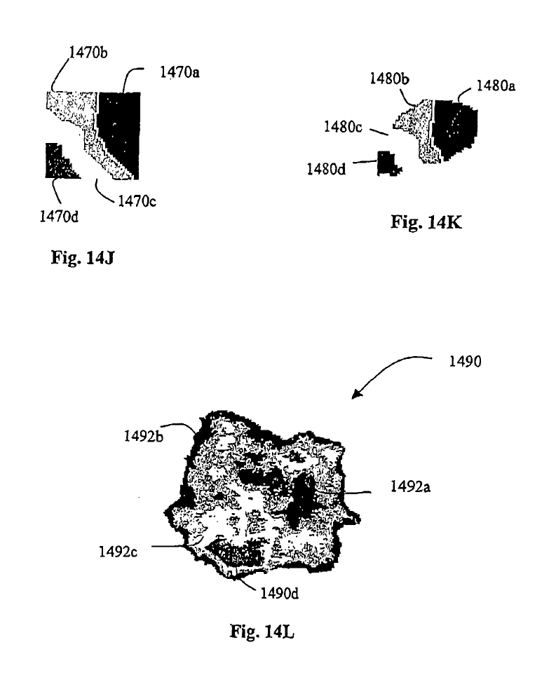

In step 506, features of lesion texture and symmetry are quantified, as further described with reference to FIGS. 10, 11 and 12. In step 508 measures of categorical symmetry of the lesion image are calculated, as further described with reference to FIGS. 13 and 14.

Although steps 502-508 are illustrated as occurring sequentially, these steps may be performed in a different order. Alternatively, if suitable computing facilities are available, steps 502-508 may be performed in parallel or as a combination of sequential and parallel steps.

The method steps 502-508 perform a variety of processes to determine a range of features relating to lesion colour, shape, texture and symmetry. Once calculated, the features are stored in memory as descriptor data 308. The features may be analysed in their raw state by a clinician (step 310), or the features may be fed into an automated classifier in order to determine whether the lesion being examined is a melanoma, a non-melanoma or a possible melanoma.

Colour Measures

Pathology indicates there exist three primary sources of colour within a melanoma, these being tissue, blood and melanin. Tissue colour is fairly clear or translucent, although by reflecting incident light it tends to make other colours less saturated. Blood contributes to a range of red colours ranging from pinks, where other reflected light affects colour, to a deeper red due to erythema, typical of the increased circulation found in lesions. Melanin occurs in a lesion in the form of small deposits of brown pigment in melanocytes, the rapidly dividing core of a melanoma cancer. The colour due to melanin can range from a light tan to a very dark brown or black. Under certain conditions, incident white light comprising detectable red, green and blue primary components may be reflected from within a melanoma and will suffer a slight attenuation of the red and green components due to the presence of blood and melanin, resulting in a bluish tinge appearing at the surface. This colour effect is termed Blue-White Veil (BWV). Medical diagnosticians usually classify lesion colours into a small range of reds, browns and blues.

FIG. 6A shows a flow chart of a method for automatically separating a lesion image into a set of colours which includes shades of brown, shades of blue and shades of red. In one arrangement fourteen colour classes are used, namely Black, Grey, Blue-White-Veil, Blue1, Blue2, Darkbrown, Brown, Tan, Pink1, Pink2, Red1, Red2, White and Skin.

In step 600 the lesion image is retrieved from memory and unwanted areas such as bubbles and hair are masked out. Next, in step 602, the value of each pixel in the wanted area, defined by its (R,G,B) coordinates, is compared with a Look-Up Table (LUT). On the basis of this comparison the pixel is allocated to one of the predefined colour classes. Repeating this step for all the pixels in the wanted area yields a segmented lesion image.

In step 604 the area of the lesion assigned to each of the colour classes is calculated. This may be expressed as a proportion of the total area allocated. In one implementation a lesion is declared to be multi-coloured if it has pixels added to at least five colour classes.

FIG. 6B illustrates a method for generating the look-up table (LUT) used in the method of FIG. 6A In step 620, a training set of lesion data is collected from lesion images captured by the apparatus and methods of FIGS. 1 to 4. In step 622, this training data is manually segmented and labelled by an expert into colours present in melanomas (black, grey, blue-white veil, white, dark brown, red and pink) and colours present in non-melanomas (brown, tan, blue, haemangioma blue, naevus blue, red, benign pink and haemangioma pink). In the following steps, the manually segmented training data are used to separate the calibrated colour space into three primary colour regions: red (containing the red and pink training data); blue (containing black grey, blue-white veil, white, blue, haemangioma blue and naevus blue training data) and brown (containing dark brown, brown and tan training data).

In step 624, the known statistical technique of canonical variate analysis (CVA) is applied to find the plane in Red-Green-Blue (RGB) colour space which best separates the Blue classes from the Non-Blue classes. Next, in step 626, CVA is used to find the plane in RGB space that best divides the Non-Blue classes into Red and Brown classes.

In step 628 the Brown class is further subdivided, This is done by applying CVA to the brown training data to establish the primarily Canonical Variate Axis (CVBrowns1). This axis is subdivided, by thresholding, to delimit shades of brown, namely Black, Darkbrown, Brown, Tan and Skin. The thresholds identified along the CVBrowns1 axis are used to identify regions in RGB space corresponding to the Black, Darkbrown, Brown, Tan and Skin classes.

In step 630 the Red class is further subdivided. This is done by applying CVA to the red training data to establish primary and secondary Canonical Variate Axes (CVRed1 and CVRed2). The two-dimensional space defined by CVRed1 and CVRed2 is divided by thresholding into areas corresponding to shades of Red, namely Red1, Red2, Pink1, Pink and Black. The areas thus defined in (CVRed1, CVRed2) space may be mapped back into RGB space to locate regions defining the Red1, Red2, Pink1, Pink2 and Black colour classes in RGB space.

In step 632 the Blue class is further subdivided. This is done by applying CVA to the blue training data to establish p ry and secondary canonical variate axes (CVBlue1 and CVBlue2). The two-dimensional space defined by CVBlue1 and CVBlue2 is divided by thresholding into areas corresponding to shades of blue, namely Blue1, Blue2, Blue-White-Veil, Grey, Black and White. The areas thus defined in (CVBlue1, CVBlue2) space may be mapped back into RGB space to locate three-dimensional regions defining the Blue1, Blue2, Blue-White-Veil, Grey, Black and White colour classes in RGB space.

In step 634 a Look-Up Table (LUT) is generated which defines the colour classes in RGB space. The LUT is constructed by applying the canonical variate transforms calculated in steps 624 to 632 to the entire gamut of RGB values, ie. all RGB values in the range [0-255, 0-255, 0-255]. Thus every possible RGB value in the range is assigned to one of the colour classes. The LUT is stored for subsequent use in segmenting lesion images as described above with reference to FIG. 6A.

The following code and canonical variate transforms show an example of the method of FIG. 6B applied to a particular set of training data. While the rules illustrate the method, the actual numbers depend on the training set used and may differ if another set of training data is used:

| | |
|---|---|
| 1. | let dataMKII = [R,G,B] values in the range [0-255,0-255,0-255] |

and %*% indicate matrix multiplication.

| | |
|---|---|
| 2. | CV1.bluvnon = dataMKII%*%bluvnon[“CV1”] |
| 3. | if (CV1.bluvnon < −0.2) { |

| | |
|---|---|
| 4. | CV1.blu = dataMKII%*%bluvblu[“CV1”] |
| 5. | CV2.blu = dataMKII%*%bluvblu[“CV2”] |
| 6. | if (CV1.blu >= −1.5) class = 1 # black |
| 7. | else if (CV1.blu < −7) class = 14 # white |
| 8. | else { |

| | |
|---|---|
| 9. | if (CV1.blu >= −4.5) { |

| | |
|---|---|
| 10. | if (CV2blu >= −1.75) class = 2 # grey |
| 11. | else class = 3 # bwv |

| | |
|---|---|
| 12. | } |
| 13. | else { |

| | |
|---|---|
| 14. | if (CV2blu < −1.75) class = 4 # blue1 |
| 15. | else class = 5 # blue2 |

| | |
|---|---|
| 16. | } |

| | |
|---|---|
| 17. | } |

| | |
|---|---|
| 18. | } |
| 19. | else { |

| | |
|---|---|
| 20. | CV1.redvbrn = dataMKII%*%redvbrn[“CV1”] |
| 21. | if(CV1.redvbrn < 3.5) { |

| | |
|---|---|
| 22. | CV1.brn = dataMKII%*%brnvbrn[“CV1”] |
| 23. | if (CV1brn < 1) class = 1 # black |
| 24. | else if (CV1.brn < 4.5) class = 6 # dk brown |
| 25. | else if (CV1.brn < 9.5) class = 7 # brown |
| 26. | else if (CV1.brn < 15) class = 8 # tan |
| 27. | else class = 15 # skin |

| | |
|---|---|
| 28. | } |
| 29. | else { |

| | |
|---|---|
| 30. | CV1.red = dataMKII%*%redvred[“CV1”] |
| 31. | CV2.red = dataMKII%*%redvred[“CV2”] |
| 32. | if (CV1.red >= 8.0) { |

| | |
|---|---|
| 33. | if (CV2.red < 1.0) class = 12 # pink1 |
| 34. | else class = 9 # pink2 |

| | |
|---|---|
| 35. | } |
| 36. | else if (CV1red < 2.0) class = 1 # black |
| 37. | else { |

| | |
|---|---|
| 38. | If (CV2.red < 1.0) class = 11 # red1 |
| 39. | else class = 10 # red2 |

| | |
|---|---|
| 40. | } |

| | |
|---|---|
| 41. | } |

| | |
|---|---|
| 42. | } |

where:

1st Canonical Variate transform to separate blues from non-blue colours is, bluvnon[“CV1”]=(R) 0.13703 (G)−0.036301 (B)−0.18002

1st Canonical Variate transform to separate blues from other blue colours is, bluvblu[“CV1”]=(R)−0.16373 (G)−0.076523 (B)0.15152

2nd Canonical Variate transform to separate blues from other blue colours is, bluvblu[“CV2”]=(R) 0.22244 (G)−0.10902 (B)−0.25107

1st Canonical Variate transform to separate reds from brown colours is, redvbrn[“CV1”]=(R) 0.11814 (G)−0.38443 (B)0.31028

1st Canonical Variate transform to separate browns from other brown colours is, brnvbrn[“CV1”]=(R) 0.24576 (G)−0.14426 (B)−0.036113

1st Canonical Variate transform to separate reds from other red colours is, redvred[“CV1”]=(R) 0.19146 (G)−0.1596 (B)0.025489, and the

2nd Canonical Variate transform to separate reds from other red colours is, redvred[“CV2”]=(R) 0.055587 (G)−0.30307 (B) 0.23504.

This process is illustrated in FIGS. 7A to 7C, which show a hypothetical histogram drawn for explanatory purposes. The methods of FIGS. 6A and 6B operate in RGB colour space. It is difficult, however, to display three-dimensional histograms. Accordingly, for ease of illustration, FIGS. 7A-C show a single plane through a trivariate RGB histogram. FIG. 7A is effectively a slice through the 3-D histogram at a fixed value of red (R). The resulting plane is defined by a blue axis 700 and a green axis 702. For each point on the plane the corresponding number of pixels occurring in the lesion is plotted. This results in a histogram 706. The peaks within the histogram 706 fall roughly into either browns or blues.

FIG. 7B shows the result of a canonical variate analysis which finds the best plane 708 that separates the brown classes 710 from the blue classes 712. Because the R dimension is not shown, the plane 708 appears as a line in FIG. 7B.

FIG. 7C illustrates the further subdivision of the brown class 712. The principal canonical variate axis (CVBrown1) 711 is found for the brown training data. The lines 718, 716 and 714 divide the brown class 712 into dark browns, browns, tan and skin respectively.

Derived Colour Variables

In addition to the colour classes described above, further derived colour variables may be calculated. These derived colours are defined as follows:

| | |
|---|---|
| Reds | Pink2 + Red2 + Red1 |
| Haemangioma | Pink2 + Red2 + Red1 + Pink1 |
| BWV blues | Grey + BWV |
| Blues | Grey + BWV + Blue1 + Blue2 |
| Blue Whites | Grey + BWV + Blue 1 + Blue2 + White |
| Tan Skin | Tan + Skin |
| Red Blues | Haemangioma + Blues |

The RedBlues colour combination was included since these normally suspicious colours can appear in benign lesions such as haemangiomas and blue naevi.

The areas of all colour variables relative to the total area of the lesion may be generated as additional features.

Measurement of Blue-White Veil

FIG. 6C illustrates a method of constructing a look-up table to assist in recognising the presence of blue-white veil (BWV) colour in a lesion.

In step 640 an expert clinician manually assembles a set of sample BWV regions from previously collected test data. Then, in step 642, a three-dimensional histogram of the BWV samples is constructed in RGB space. The training data roughly forms a normally distributed ellipsoid which may be characterised by a mean vector and covariance matrix statistics.

In step 644, a BWV region is defined in RGB space. This region is defined as the 95% confidence region of the test data, assuming a Gaussian distribution

FIG. 6D illustrates how the BWV look-up table is used in analysing a lesion.

In step 646 the colour lesion image is retrieved from memory and unwanted regions such as bubbles mid hair are masked out. Next, in step 648, the look-up table calculated using the method of FIG. 6C is used to determine the total area of BWV colour in the lesion, measured in number of pixels. This total BWV area is stored as a first value.

The presence of BWV colour is more significant in assessing a lesion if it is confluent or contiguous. In step 650 the BWV pixels are assessed to see whether any one region of BWV colour is greater than a parameter bwv_contig. If there is at least one area which meets this spatial criterion, a logical flag is set to “true”.

As a consequence of the above, a data set of colour features is obtained regarding the lesion. The data set may be used in subsequent classification.

Shape Measures

An expert diagnostician examining a lesion will typically look for a range of shape-based features which are good indicators that the lesion may be malignant. Terms such as regularity and symmetry are used, although in this context the definitions of “regularity” and “symmetry” are imprecise and fuzzy. The shape measures described below quantify features of shape and can accordingly be used in the further process of automatically classifying a pigmented skin lesion. Most of these measures are based on the fact that for physiological reasons a malignant lesion grows in a more irregular manner than a benign one.

The flow chart of FIG. 8A gives an overview of the shape measures derived in a preferred arrangement. In this flow chart, many steps are shown as occurring sequentially. However, in many cases it is possible to perform these measures in a different order, in parallel, or in a combination of parallel and sequential steps.

Firstly, in step 800, a segmented binary image of the lesion is obtained from memory. Next in step 802, the area, perimeter, width, length and irregularity of the lesion are calculated. An example of a segmented lesion image is shown in FIG. 9A, in which the contour 900 defines the boundary of the lesion and is typically derived from the border detection process 412 of FIG. 4. The area within the boundary 900 is designated as being lesion, while the area outside the boundary 900 is designated as being skin. The lesion boundary 900 is drawn with respect to the coordinate system defined by an x-axis 902 and an orthogonal y-axis 904. An enclosing rectangle 906 is drawn to touch the extremities of the boundary 900. The length and width of the enclosing rectangle 906 are stored as features of the lesion.

The area of the lesion (AREA) is estimated by the number of pixels assigned to the binary image of the segmented lesion, ie. the area enclosed by the boundary 900. Likewise, the perimeter measure (Perimeter) is estimated from the number of pixels assigned to the boundary 900. The ‘border irregularity’ parameter is then determined as follows to give an indication of the departure of the lesion shape from that of a smooth circle or ellipse:

$$Border irregularity = \frac{{Perimeter}^{2}}{4 π . AREA} .$$

In step 804 of the shape analysis, the structural fractal measurement S3 is obtained. This parameter is a measure of the difference between the lesion boundary 900 and a smooth boundary: the rougher the boundary the higher the fractal dimension. This parameter will be described in greater detail with reference to FIG. 8C.

In step 806 an analysis of the “notches” or small concavities in the lesion boundary is conducted. This analysis will be described in more detail with reference to FIG. 5B and FIGS. 9C and 9D.

In step 807, edge abruptness measures are calculated. This is described in more detail with reference to FIG. 5D and FIGS. 9E-9F. The edge abruptness calculation requires not only the binary lesion mask but also grey-level information.

In step 808 of FIG. 8A, a best-fit ellipse is fitted to the shape of the lesion 900.

This is illustrated in FIG. 9B in which the dotted boundary 908 shows the ellipse which is a best fit to the lesion boundary 900.

In order to calculate the best-fit ellipse, the binary centre of mass (\overline{x},\overline{y}) of the lesion is calculated from the following equations:

$$\begin{matrix}\overline{x} = \frac{1}{AREA} {\sum }_{( x , y )}^{} {\sum }_{e ℜ}^{} x \\ \overline{y} = \frac{1}{AREA} {\sum }_{( x , y )}^{} {\sum }_{e ℜ}^{} y\end{matrix}$$

The summation is over the binary lesion mask [symbol] , ie. all pixels (x,y) in the lesion area with unwanted regions such as hair and bubbles removed. The central moment of order (p,q) of the lesion is defined by:

$${μ}_{pq} = {\sum }_{( x , y )}^{} {\sum }_{e ℜ}^{} {( x - \overline{x} )}^{p} {( y - \overline{y} )}^{q}$$

The binary centre of mass and central moments of the lesion are then used to calculate the length of the major axis a of the best-fit ellipse, the length of the minor axis b of the ellipse and the orientation of the ellipse. These terms are illustrated in FIG. 9B which shows the best-fit ellipse 908 with respect to a new set of axes defined by an a-axis 910 and an orthogonal b-axis 912. The a-axis 910 runs along the length of the ellipse and the b-axis 912 constitutes the minor axis of the ellipse 908. The orientation θ of the ellipse is defined as the angle between the original x-axis 902 and the a-axis 910.

The length of the best-fit ellipse along the major axis a is obtained from:

$$a = \sqrt{\frac{( {μ}_{20} + {μ}_{02} ) + \sqrt{{( {μ}_{20} + {μ}_{02} )}^{2} + 4 {μ}_{11}^{2}}}{\frac{\sqrt{π}}{4} {( {μ}_{20} {μ}_{02} - {μ}_{11}^{2} )}^{\frac{1}{4}}}}$$

The greatest length of the ellipse along the minor axis b is obtained from:

$$b = \sqrt{\frac{( {μ}_{20} + {μ}_{02} ) + \sqrt{{( {μ}_{20} + {μ}_{02} )}^{2} + 4 {μ}_{11}^{2}}}{\frac{\sqrt{π}}{4} {( {μ}_{20} {μ}_{02} - {μ}_{11}^{2} )}^{\frac{1}{4}}}}$$

The orientation θ of the ellipse is calculated from:

$$θ = \frac{1}{2} {tan}^{- 1} ( \frac{2 {μ}_{11}}{{μ}_{20} - {μ}_{02}} ) .$$

The calculation of the centre of gravity, length of the major and minor axes and the orientation of the ellipse constitute step 810 of the flow chart of FIG. 8A.

Next, in step 812 a bulkiness parameter is calculated for the lesion. The bulkiness parameter is defined by the area of the best-fit ellipse divided by the area of the lesion:

$$bulkiness = \frac{π . a . b}{AREA} .$$

In step 814, a measure of the symmetric difference of the lesion about the major axis 910 or the minor axis 912 of the ellipse 908 is obtained from:

$${Sym}_{l} = 100 \frac{\# \{ ℜ / {ℜ}_{l} \}}{AREA}$$

In this equation the index i indicates either the major axis 910 (i=1) or the minor axis 912 (i=2). The lesion image is [symbol] , and [symbol] ₁ is the reflection of the lesion across axis i. The term having the form X/Y indicates the difference between sets X and Y, and # represents cardinality or area. The resultant measure is percentage normalised with respect to the area of the lesion.

Analysis of Outline Notches

Notches are small concavities in the boundary of the lesion. The number and extent of these boundary irregularities may provide an indication of a malignant lesion. A series of notch measures is obtained from a morphological analysis of the periphery of a lesion, as shown in FIG. 8B. In step 820 the binary lesion image is extracted from memory. This is illustrated in FIG. 9C, in which the shape 920 is an example of a binary lesion image having notches in its perimeter. In step 822 the binary lesion image 920 is subjected to the standard morphological operation of closing. In a preferred arrangement, the structuring element used for the closing is a disc of radius 31 pixels. The region 922 in FIG. 9C is the result of the closing operation performed on lesion image 920. The closing operation has acted to “fill in” the notches in the boundary of the lesion 920.

In step 824 the difference between the closed region 922 and the original lesion image 920 is calculated. In the example of FIG. 9C the differencing operation results in the four shapes 924a-d. The shapes 924 correspond to indentations in the original image 920. Steps 822 and 824 may be summarised in the following equation, in which φ_{ρ(31)} is the known morphological operation of closing with a disc of radius 31 pixels and [symbol] is the binary lesion image:

Next, in step 826, the identified regions 924 are subjected to the standard morphological operation of opening. In a preferred arrangement, the structuring element used is a disc of radius 2 pixels. The opening operation removes noise and long thin filament type structures and results in the shapes 926a-c seen in FIG. 9C. Step 826 may be summarised in the following equation, in which γ_{ρ(2)} is the standard morphological operation of opening with a disc of radius 2:

B=γ_{ρ(2)}(A).

The radius parameters 2 and 31 are optimal values derived from experimental work and may be changed appropriately if image size or image quality is altered.

Finally, in step 828 each of the remaining notch areas 926 is measured. The area n_{λ} of each notch 926 is measured by counting pixels. The notch depths nₗ are measured as the morphological geodesic distance from the outer edge of the mask 920 to the point furthest inwards towards the centre of the mask 920. The width n_{w} of each notch 926 is calculated as:

n_{w}=n₀/nₗ.

Each notch 926 is then assessed using a test derived from research work conducted with an expert dermatologist:

$$\begin{matrix}{n}_{s} = 0.003596935 {n}_{a} - {0.139300719}_{{n}_{l}} - \\ {0.127885580}_{{n}_{w}} + 1.062643051 \frac{{n}_{w}}{{n}_{i}}\end{matrix}$$

where the resulting parameter n_{δ} represents notch significance. All notches with a significance n_{δ}<1.5 are considered significant. For all the significant notches associated with a lesion, the following measurements are collected:

NN number of notches;

MND mean notch depth;

MW mean notch width;

MECC mean eccentricity, being n_{w}/nₗ;

LGD largest notch depth;

LNA largest notch area; and

LECC largest notch eccentricity.

FIG. 9D illustrates the calculation of the length of a notch. The shape 940 corresponds to the notch 926a of FIG. 9C with contours indicating the geodesic distance from the outside edge 944 of the notch. The arrow 942 indicates the furthest distance to the outside edge 944.

Structural Fractal Measurement S3

Fig. 8C is a flow chart which shows in more detail a preferred method of calculating the fractal dimension of the lesion boundary (ie. step 804 of FIG. 8A).

A smooth boundary is noticeably different from a rough one, and a measure of this difference may be found in the fractal dimension of the boundary. Typically the rougher the boundary the higher the fractal dimension. In a preferred arrangement this may be measured by correlating changes in the perimeter with the amount of smoothing done to the perimeter shape.

In step 862 the lesion boundary is retrieved from memory. Next, in step 864 a disc is initialised to act as a structuring element. In step 866 the lesion boundary is subjected to the standard morphological operation of dilation with the disc of radius r. In the following step, 868, the width of the curve is 2r and the area of the curve is A, given by the number of pixels making up the dilated curve. A preferred estimate of the length (l) of the dilated boundary is:

l=A/2r.

In the following step 870, the radius r is increased. Step 872 is a decision step to check whether the radius r has reached an upper bound b. If r is less than b the method returns to step 866 and the dilation is performed once more with a disc having an increased radius. If in step 872 it is found that r is greater than b (the YES output of step 872), the method continues to step 874, in which the length l of the dilated boundary is plotted on log-log axes as a function of radius r. In the final step 876, the slope of the plotted curve is calculated. The S3 measure is the slope of this plot. From the analysis of many such curves, it was determined that the most discriminating part of the curve for distinguishing between melanoma and non-melanoma was the slope of the line obtained between τ=10 pixels and r=20 pixels. The part of the curve where τ is less than 10 is not of much use because it can be dominated by noise. Also, the part of the curve where τ is greater than 20 was found to be unreliable because the smallest lesions generally have an average radius not much greater than 20 or 30 pixels. Accordingly the measure S3 is taken to be the measure of slope between r=10 and r=20 and represents the fractal dimension of the boundary.

Outline Edge Abruptness

If the edge of the lesion is not abrupt but rather fades into the surrounding skin, the underlying cell behaviour is different from those cases where there is an abrupt transition from the lesion to the surrounding skin. A clinician will consider the lesion/skin transition in assessing a lesion. A method of quantifying this transition is outlined in the flow chart of FIG. 8D and illustrated in FIGS. 9E and 9F.

In step 840 the grey-scale lesion image and binary lesion mask are retrieved from memory and in step 842 the lesion boundary is obtained. Next, in step 844, the region within the boundary is divided into a set of inner contours. In step 846 the region outside the boundary is divided into a set of outer contours. This is illustrated in FIG. 9E which shows a lesion boundary 940 together with two inner contours 944a and 944b and two outer contours 942a and 942b. A standard morphological distance transform is used to generate the inner and outer contours 942 and 944. A range of distance of up to 51 pixels inside the boundary and a distance of 51 pixels outside the boundary may be considered.

In step 848 an edge profile is calculated. This is done by obtaining the mean grey level value along each of the contours 940, 942, 944 considered. The edge profile is normalised in step 850 such that the maximum value is 1 and the minimum value is 0. An edge profile 954 is shown in FIG. 9F where the x-axis 950 is a distance measure and the y-axis 952 indicates the normalised mean grey-level value corresponding to each x value. The x ordinate that is associated with the edge profile value of 0.5 is used to define a mid-point 956 of the profile. The range of 10 pixels (dx) on either side the mid point defines two areas calculated in step 852. The left shoulder region 960 has an area S₁ and the right hand shoulder 958 has an area Sᵣ. Finally, in step 854, the edge abruptness measure is obtained from the equation:

EA=S₁+Sᵣ.

The larger the parameter EA, the more abrupt the transition from lesion to skin.

The method described above provides an edge abruptness measure averaged over the entire lesion. Edge abruptness measures may also be calculated for different portions of the lesion boundary. In an alternative arrangement the lesion is divided into four quadrants and the edge abruptness of each quadrant is calculated. The four edge abruptness measures thus obtained are compared and the differences noted. Large differences between two or more of the four measures may be indicative of a melanoma.

As a consequence of the above, a data set of shape features is obtained regarding the lesion. The data set may be used subsequently for lesion classification.

Texture And Symmetry Measures

Clinicians use a number of observed features of texture and symmetry to establish their diagnosis. One important characteristic of melanoma is the notion of regularity. Melanomas are irregular skin lesions. The measures discussed below with reference to FIG. 10A seek to quantify the regularity of a lesion

FIG. 10A shows a series of steps performed to calculate symmetry and texture measures of a lesion. Not all the steps need be performed in the sequence indicated. For example, steps 1002, 1004 and 1006 may be calculated in a different order, in parallel or in a combination of parallel and sequential steps.

In step 1000 the grey-level lesion image and binary lesion mask are retrieved from computer memory. In step 1002 the “variance”, “network”, “darkblob” and “borderspots” values are calculated.

The variance measure evaluates the quantity of high-frequency information present in a lesion. A preferred method is to apply an edge-preserving low-pass filter to the lesion image and then to output a root-mean-square (RMS) of the difference between the original image and the filtered image, calculated over the lesion area.

The edge-preserving low-pass filter is preferably a morphological levelling, that is, an alternating sequential filter followed by grey-level reconstruction.

Let asfᵣ be the low-pass filter with a convolution mask of radius r, A the area of the lesion and I(x,y) the grey-level of the wanted area of the lesion image at location (x,y), that is, excluding bubbles and hairs. Then J=asfᵣ(I(x,y)) and the variance measure V can be expressed as:

$${V}^{2} = \frac{1}{A} {\sum }_{A}^{} {( I ( x , y ) - J ( x , y ) )}^{2}$$

where the summation over the lesion area is restricted to that clear of hairs and bubbles.

If the lesion as a whole is relatively smooth, the variance measure will be low, and if it has a lot of high-frequency information the measure will be high. Benign lesions tend to be smoother.

The “network” measure is also calculated in step 1002. A network consists of a pattern of dark pigment across the lesion interconnecting to form a mesh, and is an indicator of the underlying melanocytes which are now active. The network is segmented by looking at the complement of dark network, ie. the small white domes that are surrounded by network. These white dots are detected as follows on the lesion area of an image I, with hairs and bubbles masked out:

A=(I−γ′(I))>α₁

B=(I−γ^{r}(I))>α₂

C=ρ(A,B)

where γ′ is an opening by reconstruction, > is a threshold operator, α₁ is larger than α₂ and ρ is the binary reconstruction operator. A morphological closing is performed on the result C within the wanted region of interest (ie. removing hair and bubbles) to bring together white domes that are close to one another. The area which results from this operation is the network measure. It is expressed as a percentage of the lesion area

The third measure calculated in step 1002 is “darkblob”, which is similar to the network measure. It looks for light linear structures on a dark background. For a lesion image I:

A=(φ^{r}(I)−I)>α₃

B=(φ^{r}(I)−I)>α₄

C=γₛ^{α}(ρ(A,B))

where φ^{r} is a closing by reconstruction, > is a threshold operator, α₃ is larger than α₄, γₛ^{α} is an opening by area with parameter s, and ρ is the binary reconstruction operator.

Within the wanted region of interest, ie without hair or bubbles, morphological closing is performed on the results of these operations to bring together the round dark structures that are spatially close. The area which results, as a percentage of the lesion area, is the darkblob1 measure. The number of blobs found is the darkblob2 measure.

The final measure calculated in step 1002 is the “borderspots” variable. This measure detects peripheral black dots and globules, which are symptomatic of a growing lesion. The approach is similar to that used in the darkblob measure but the search is restricted to the peripheral or border region and large spots are rejected. The border region is defined as follows:

Border=δₚ(δ₁(M)∩^{M}))∩M

where M is the binary lesion mask image, δ₁ is the dilation of radius 1 and δₚ is a dilation of radius p. Border is a mask of the area in which darkblobs are searched for.

Next the following steps are performed:

A=(φₚ₁^{r}(I)−I)>α₇

B=(φₚ₁^{r}(I)−I)>α₈

C=γₛ₁^{α}(ρ(A, B)).

The thresholds α₇ and α₈ are used to detect round spots.

These operations are repeated, but with different radius p₂), area opening parameter (s2) and thresholds (α₉ and α₁₀) in order to detect bigger spots:

D=(φₚ₂^{r}(I)−I)>α₉

E=(φₚ₂^{r}(I)−I)>α₁₀

F=γₛ₂^{α}(ρ(D,E)).

The larger spots are then excluded from the set:

G=C∩F

The resulting measure is the area of detected dark spots within the region of interest defined by Border. This is weighted by the inverted grey values for those spots, such that the darker the spots the higher the measure.

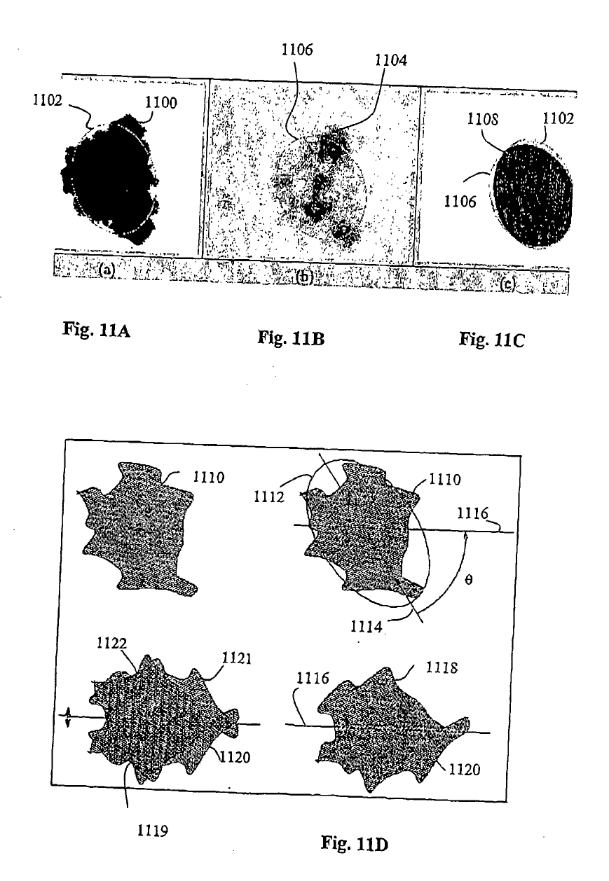

In step 1004 of FIG. 10A the symmetry of the binary lesion mask is compared with that of the grey-level weighted or colour weighted symmetries of the lesion. The resulting measures of angular difference and grey-level difference are discussed in more detail with reference to the flow chart of FIG. 10B and FIG. 11A.

In step 1006 “image flip” measures are calculated which assess large-scale symmetries by first finding the centre and axis of symmetry of the lesion and then comparing pixel values symmetrically across the centre and axis. These measures will be described in more detail with reference to the flow chart of FIG. 10C mid FIG. 11B.

The next series of measures may be characterised as quantifying radial symmetry. In step 1008, the lesion mask retrieved in step 1000 is divided into radial segments that are centred on the centroid of the lesion. This radial segmentation is described in more detail with reference to FIG. 10D. Following the segmentation performed in step 1008, the “radial mean difference”, “radial mean point wise variance” and “simple radial variance” are calculated in step 1010.

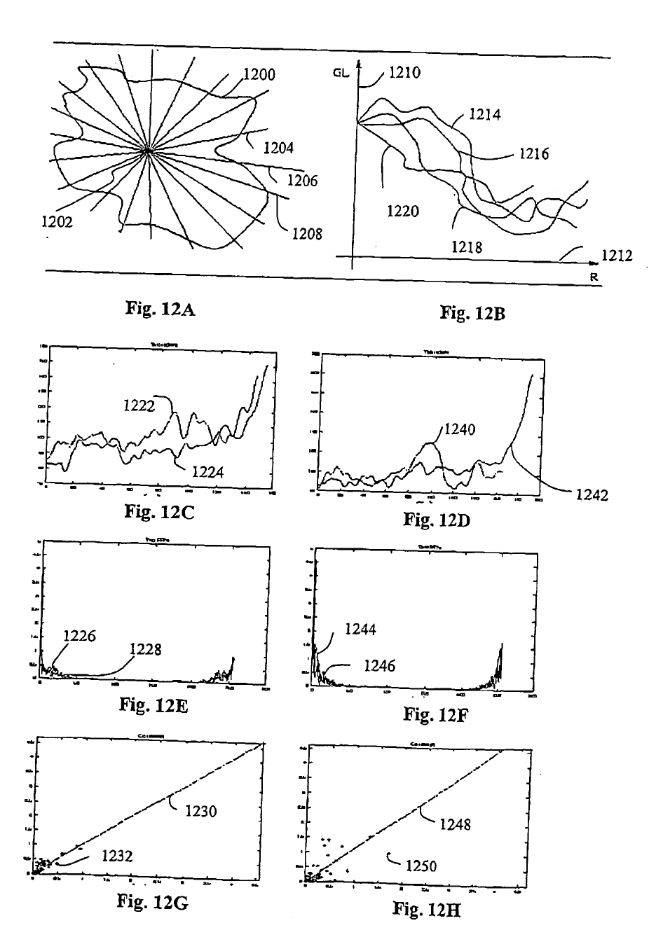

In step 1012, the Fourier Transform of each radial segment is calculated. Next, in step 1014, the Fourier Transforms of each of the radial segments are compared and a set of measures generated. These will be described in more detail with reference to FIG. 10D and FIG. 12.

FIG. 10B is a flow chart which describes in more detail the comparison of the binary and grey-scale best-fit ellipses. The steps of FIG. 10B correspond to step 1004 of FIG. 10A.

In step 1020 the lesion image is retrieved from memory. Then, in step 1022 the ellipse that is the best fit to the binary image mask is calculated. This may be done using the method of step 808 or it may be calculated by the following moment-based method.

Order n moments are computed in the following manner:

$${μ}_{ij} = {\sum }_{( x , y ) ∈ ℜ} {x}^{i} {y}^{j}$$

for pixels (x,y) in the binary lesion mask [symbol] , where i+j=n. The best-fit ellipse parameters are obtained by computing the eigenvectors \overline{ν₁} and \overline{ν₂} and the eigenvalues λ₁ and λ_{the order-}2 matrix M₂:

$${M}_{2} = [ \begin{matrix}{μ}_{20} & {μ}_{11} \\ {μ}_{11} & {μ}_{02}\end{matrix} ]$$

Then A is the area of the best-fit ellipse and the major and minor axes a and b are given by:

A=\sqrt{πλ₁λ₂} and

$$a = \sqrt{\frac{{λ}_{1}}{A}} and b = \sqrt{\frac{{λ}_{2}}{A}} .$$

An example is shown in FIG. 11A, which shows a binary lesion mask 1100 and a best-fit ellipse 1102 matched to the binary lesion mask 1100.

The same moments-based method can be used with the grey-level value of each pixel, g(x,y) as a weighting function. The weighted moments are given by.

$${μ}_{ij}^{g} = {\sum }_{( x , y ) ∈ ℜ} g ( x , y ) {x}^{i} {y}^{j} .$$

The eigenvectors ν_{g1} and ν_{g2} of the grey-level weighted order 2 moments matrix define the major and minor axis of the grey-level freighted best-fit ellipse.

This is illustrated in FIG. 11B, which shows a grey-level lesion image 1104 together with a best-fit ellipse 1106 calculated using grey-level weighted moments. When the grey levels are evenly or symmetrically spread across a lesion, there should be little discrepancy between the binary and the grey-level weighted result. FIG. 11C shows the binary best-fit ellipse 1102 superimposed on the grey-level best-fit ellipse 1106. The area 1108 shows the intersection of the two best-fit ellipses 1102, 1106.

Next, in step 1026, the angular difference between the binary best-fit ellipse and the grey-level weighted best-fit ellipse is calculated. The angular difference measure is the angle in radians between the eigenvectors ν of the binary best-fit ellipse and the eigenvectors ν_{g} of the grey-level best-fit ellipse. The angular difference is calculated as follows:

$$A D = {cos}^{- 1} ( \frac{\overline{{v}_{g1}} \cdot \overline{{v}_{1}}}{‖ \overline{{v}_{g1}} ‖ \cdot ‖ \overline{{v}_{1}} ‖} ) .$$

The more symmetrical the lesion, the closer the angular difference will be to zero. In practice it is necessary to allow for possible π/2 offsets to handle cases where the lesion area is nearly circular, This is done by taking the remainder of the angular difference after division by π/4.

Next, in step 1028, a “grey level difference” (GLDdiff) is calculated. This is the difference between the centroids of the binary best-fit ellipse and the grey-level best-fit ellipse. The distance is normalised for the area of the lesion, and may be calculated from the order n moments defined above as follows:

$${δ}_{g} = \sqrt{{( \frac{{μ}_{10}}{{μ}_{00}} - \frac{{μ}_{10}^{g}}{{μ}_{00}^{g}} )}^{2} + {( \frac{{μ}_{01}}{{μ}_{00}} - \frac{{μ}_{01}^{g}}{{μ}_{00}^{g}} )}^{2}}$$

$$GLDdiff = {δ}_{g} \sqrt{\frac{2}{{μ}_{00} + {μ}_{00}^{g}}} .$$

Image Flip Measures

The flow chart of FIG. 10C gives more detail of the calculation of the image flip measures, which quantify how similar with itself a lesion image is when flipped about an axis of symmetry.

In step 1030 the lesion image is retrieved from memory. Next, in step 1032, the axes of symmetry of the lesion are found. This may be done by using the weighted-moment method described above.

An example of the method of FIG. 10C is shown in FIG. 11D. A grey-weighted best-fit ellipse 1112 is fitted to a lesion mask 1110. The ellipse 1112 defines a major axis 1114 of the lesion 1110. The lesion 1110 is portrayed in a space defined by the horizontal axis 1116. The angle between the major axis 1114 of the ellipse 1112 and the horizontal axis 1116 is defined to be θ.

Next, in step 1034 the lesion image 1110 is rotated such that the major axis 1114 coincides with the horizontal axis 1116. This is illustrated in FIG. 1D in which, following rotation of the lesion image 1110, the lesion has an area 1118 above the horizontal axis 1116 and an area 1120 which is below the horizontal axis 1116. Denote the rotated image as I.

Next, in step 1036, the image I is flipped about the horizontal axis 1116 to give shape Iₕ. This is illustrated in FIG. 11D in which area 1121 is the mirror image of area 1120, formed above the horizontal axis 1116. Area 1119 is the mirror image, formed below the horizontal axis 1116, of the area 1118. Area 1122 is the intersection of I and Iₕ.

In step 1038 the Fₕ measure is calculated as follows:

$${F}_{h} = \frac{1}{AREA} \sqrt{{\sum }_{( x , y ) ∈ ( I ⋂ {I}_{h} )} {( g ( x , y ) - g ( x , - y ) )}^{2}} .$$

The weighting function g(x,y) is preferably the luminance value L. However, other values may be used such as:

L⁻¹ the inverse of the luminance component, ie. 255−L;

R the red component of the image;

R⁻¹ the inverse red component, ie. 255−R;

G the green component,

G⁻¹ the inverse green component, ie. 255−G;

B the blue component;

B⁻¹ the inverse blue component.

Additionally the weighting function image may be blurred or averaged over an octagon of radius r. The image data may also be subjected to a non-linear stretch to emphasise dark features.

In addition, three new variables were constructed from the weighting measures:

Mn the minimum value among (L,L⁻¹,R,R⁻¹,G,G⁻¹,B,B⁻¹);

med the median value among (L,L⁻¹,R,R⁻¹,G,G⁻¹,B,B⁻¹); and

Mx the maximum value among (L,L⁻¹,R,R⁻¹,G,G⁻¹,B,B⁻¹).

Next, in step 1040, the image I is flipped about the vertical axis to form Iᵥ. In step 1042 the Fᵥ(flip over vertical axis) measure is calculated by an equation analogous to that given for Fₕ but using g (−x,y) instead of g (x,−y).

A measure F_{τ} may be calculated by rotating the image I by 180°. F_{τ} is calculated by an equation analogous to that given for Fₕ. The weighting function g(−x, −y) is used instead of g (x, −y).

Three further measures may be derived from Fₕ, Fᵥ and F_{τ}:

Flipmax is the maximum of Fₕ, Fᵥ and F_{τ};

Flipmin is the minimum of Fₕ, Fᵥ and F_{τ}; and

Flipmean is the mean of Fₕ, Fᵥ and F_{τ}.

Radial Measures

The flow chart of FIG. 10D shows how aspects of the radial symmetry of the lesion may be quantified In step 1050, the lesion image is retrieved from memory and unwanted areas such as bubbles and hair are masked out. Next, in step 1052, the grey-weighted centroid of the lesion image is found as follows:

$${\overline{x}}_{g} = \frac{1}{AREA} {\sum \sum }_{( x , y ) ∈ ℜ} g ( x , y ) \cdot x {\overline{y}}_{g} = \frac{1}{AREA} {\sum \sum }_{( x , y ) ∈ ℜ} g ( x , y ) \cdot y$$

where \overline{x}_{g} and \overline{y}_{g} are the coordinates of the centroid. The grey-level weighting function g can be one of the colour components (R, G or B), their inverses, the luminance component L or the inverse of luminance.

Next in step 1054 the lesion is divided into N pie-like radials segments issuing from the lesion centroid. This is illustrated in FIG. 12A, which shows a lesion boundary 1200 having a centroid at point 1202. A series of radial lines (for example 1204, 1206, 1208) is drawn at equally spaced angular intervals. Lines 1204 and 1206 define one pie-like segment, while lines 1206 and 1208 define a second pie-like segment of the lesion 1200.

In step 1056 the image data g is accumulated along each radial segment, creating N 1-dimensional (1-D) signals. Within each radial segment the data is averaged at each distance r from the centroid such that all the pixels of the image are covered and there is no overlap between adjacent segments. The number of radial segments used is not critical. In a preferred version there is one radial segment per degree, ie. 360 radials in total. Missing data (due, for example to hairs or bubbles) is interpolated.

Examples of the radial signals are shown in FIG. 12B in which the x-axis 1212 is the radial distance from the centroid and the y-axis 1210 represents the image value g. Four radial signals 1214, 1216, 1218, 1220 are shown for purposes of illustration.

In step 1058 the “radial mean difference”, “radial mean point wise variance” and “simple radial variance” are calculated for the radial signals 1214, 1216, 1218, 1220.

The simple radial variance measure is the variance of all the radial data. Invalid data (due to hair or bubble) is omitted in the calculation. Let gᵢ be the grey level at distance i from the centroid and M be the number of valid data points along all radials, then the simple radial variance is calculated from:

$$S Var = \frac{1}{M} {\sum }_{i = 1}^{M} {g}_{i}^{2} - {( \frac{1}{M} {\sum }_{i = 1}^{M} g )}^{2} .$$

The radial mean difference measure is obtained by computing the mean value of each of the radial signals and then computing the variance of all these mean values:

MeanDiff=V(E(gⱼ))

where the gⱼ are all the valid data points along radial j.

The radial mean pointwise variance measure quantifies the variance among all radial signals at a certain distance from the centroid. This generates as many variance values as there are points in the longest radial signal. The mean value of all these variances constitutes the pointwise variance measure. If nᵢ is the number of valid radial points at distance i from the centroid (omitting hair and bubbles) and N is the number of points in the longest radial, then:

$$M = {\sum }_{i = 1}^{N} {n}_{i}$$

$$Pt Var = \frac{1}{M} {\sum }_{i = 0}^{N} {n}_{i} [ \frac{1}{{n}_{i}} {\sum }_{j = 1}^{{n}_{i}} {g}_{i , j}^{2} - {( \frac{1}{{n}_{i}} {\sum }_{j = 1}^{{n}_{i}} {g}_{i , j} )}^{2} ]$$

where gᵢⱼ is the grey level along radial j at distance i from the centroid.

In step 1060 the Fourier Transform of each radial signal is found. The Fast Fourier Transform (AFT) is used because of its known computational efficiency. Prior to finding the FFT, the N radial signals are windowed and zero-padded to the length of the longest signal. The N signals are also rounded up to the nearest integer M that cm be decomposed in products in powers of 2, 3, 5 and 7 (for Fast Fourier Transform efficiency). Windowing means imposing a smooth variation on the signal so that it tapers to 0 at each extremity. This is useful because the FFT assumes that the signal is periodic. Zero padding, which means filling the extremities of the signal length with zeros, is required since non-zero extremities ill the signal produce strong artificial peaks in the Fourier spectrum.

In step 1062 robust correlation is performed on the logarithm of the amplitude of pairs of Fourier spectra. This enables a comparison of the spectra in a translation-independent manner. The spectra are compared two by two, and a goodness-of-fit measure is calculated.

Let h and g be two spectra digitised over m samples:

h[h₁,h₂,h₃, . . . hₘ]and g=[g₁,g₂,g₃, . . . gₘ]

A robust correlation is performed by finding b such that S is minimised,

$$S = {\sum }_{i = 1}^{M} ‖ {g}_{l} - b \cdot {h}_{i} ‖$$

the goodness-of-fit measure r is given by:

r=S/m.

The smaller r is, the better the fit.

Step 1062 is illustrated in FIG. 12C to FIG. 12H. FIG. 12C shows two radial signals 1222, 1224. FIG. 12E shows the Fourier spectra 1226, 1228 of radial signals 1222, 1224. FIG. 12G shows the correlation between the Fourier spectra 1226, 1228. The crosses in FIG. 12G, for example cross 1232, are data points of spectrum 1226 plotted against spectrum 1228, The line 1230 is the best-fit line drawn through the points 1232. There is a relatively good correlation between spectra 1226 and 1228.

FIGS. 12D to 12H show a corresponding example where the spectra are less well correlated. FIG. 12D shows two radial signals 1240 and 1242. FIG. 12F shows the Fourier spectra 1244 and 1246 of the radial signals 1240 and 1242. FIG. 12H is a scatter plot of Fourier spectrum 1244 plotted against Fourier spectrum 1246. The line 1248 is the best-fit line drawn through the data points 1250, which are more widely scattered than the points 1232 shown in FIG. 12G.

In step 1064 a range of measures is generated for the set of Fourier spectra.

FFTbest

The spectrum for a radial segment i is robustly correlated with all the spectra in the 10 degree segment opposite the segment i. When 1 degree segments are used this means there will be 10 correlations for each segment i. The goodness-of-fit measures r for all robust correlations (as defined above) are sorted and the best (ie. smallest) 1% is retained as FFTbest.

FFTworst

This is calculated as for FFTbest except that the values determining the worst (ie. largest) 1% is retained as FFTworst.

FFTmed

This is calculated as for FFTbest, but this time the median value (50% value) is retained to constitute the FFTmed measure.

FFTmean

This is calculated as for FFTbest, but this time all the r values are averaged. The average value constitutes the FFTmean measure.

FFTglobalmed

All the radials are compared two by two. The goodness-of-fit measures r are sorted and the median value (50% value) is retained. This measure constitutes the FFTglobalmed measure.

FFTglobalmean

This is calculated as for FFTglobalmed but in this case all the r values are averaged. The average value constitutes the FFTglobalmean measure.

Categorical Symmetry Measures

FIG. 13A shows how further measures of symmetry may be quantified for the lesion image. The symmetry measures are derived from categorised regions within the lesion image.

In step 1300 the lesion image is retrieved from memory. In the following step, step 1302, the lesion image is segmented into regions having similar content. The measure which determines whether regions have similar content can be colour or texture based or a combination of both The resulting image of labelled regions is called a category image. Further detail regarding the segmentation of the image into a category image will be given with reference to FIGS. 13B-13C.

The act of segmenting the lesion image into categorised regions does not in itself yield any measure of symmetry. However, measures quantifying symmetry or regularity can be derived from the arrangement of segmented regions within the lesion mask. The segmentation of the image is illustrated schematically in FIG. 14A. A lesion is defined by the area within the boundary 1400. The lesion area 1400 has a centre of gravity at point G0. The two areas 1402a and 1402b are placed in the same category according to a selected measure. Regions 1402a and 1402b have a centre of gravity at point G2. Similarly, regions 1403a and 1403b are assigned to another category. Regions 1403a and 1403b have a centre of gravity at point G3. Region 1404 is allocated to a further category, which has a centre of gravity at point G4. Similarly area 1405 is allocated to a fifth category which has a centre of gravity at point G5. The lesion area 1401, which is the area inside the boundary 1400 but excluding the regions 1402a-b, 1403a-b, 1404 and 1405, has a centre of gravity at point G1.

In step 1304 the Euclidean distance between the centres of gravity of some or all of the regions is calculated.

If a and b are two distinct segmented regions, let D_{(a,b)} be the Euclidean distance between their centres of gravity. For convenience, his distance may be regarded as the distance between regions. If N regions are present, there are N(N-1)/2 such distances.

In the example of FIG. 14, there would be 10 such different distances. These distances can be ordered and relabelled such that D₁ is the largest distance and Dₜ is the smallest, where l−N(N−1)/2. The following five measures can then be calculated:

$$CS1 = \frac{1}{\sqrt{A}} {D}_{1}$$

$$CS2 = \frac{1}{\sqrt{A}} {D}_{1}$$

$$CS3 = \frac{1}{\sqrt{A}} {D}_{\frac{1}{2}}$$

$$CS4 = \frac{1}{l \sqrt{A}} {\sum }_{i = 1}^{l} {D}_{i}$$

$$CS5 = \frac{1}{\sqrt{A}} {\sum }_{i = 1}^{l} \frac{1}{i} {D}_{i}$$

where A is the total surface area of the lesion and D_{1/2} is the median distance.

CS1 is the furthest distance between two regions within the lesion. The measure is weighted by the geometric length (square root of the area) of the lesion in order to obtain a dimensionless number, CS2 is the closest distance between two distinct regions within the lesion CS3 is the median distance of all the regions within the lesion CS4 is the mean distance between regions and CS5 is a sum weighted by an arithmetic progression to give a larger importance to the greater distances with respect to the smaller. Unlike measures CS1 to CS4, CS5 increases with the number of regions, although each new region counts for less.

Next, in step 1306, area-weighted distances between regions are calculated. The previous set of measures CS1 to CS5 has the feature that even a single-pixel region has as much importance as a region half the size of the lesion. In the weighted distance measures described below, a larger region will make a larger contribution towards the measure.

If D_{(a,b)} is the distance between region a and region b as defined above, and if A(a) and A(b) are the surface area of a and b respectively, we call A′_{(a,b)} the smaller of A(a) and A(b), ie.:

$${A}_{( a , b )}^{′} = \{ \begin{matrix}A ( a ) & if A ( a ) \leq A ( b ) \\ A ( b ) & otherwise\end{matrix}$$

Each distance can now be weighted using this definition, ie.: let

D′_{(a,b)}=D_{(a,b)}A′_{(a,b).}

As before all the weighted distance between regions can be ordered and relabelled from largest to smallest, such that D′₁ is the largest weighted distance and D′₁ with

$$l = ( \frac{N \cdot ( N - 1 )}{2} )$$

being the smallest. We can now define a new series of measures:

$${CS1}^{′} = \frac{1}{{A}^{3 / 2}} {D}_{1}^{′}$$

$${CS2}^{′} = \frac{1}{{A}^{3 / 2}} {D}_{1}^{′}$$

$${CS3}^{′} = \frac{1}{{A}^{3 / 2}} {D}_{\frac{1}{2}}^{′}$$

$${CS4}^{′} = \frac{1}{1 {A}^{3 / 2}} {\sum }_{l = 1}^{l} {D}_{l}^{′}$$

$${CS5}^{′} = {A}^{3 / 2} {\sum }_{i = 1}^{l} \frac{1}{i} {D}_{i}^{′}$$

In this series of measures, the coefficient

$$\frac{1}{{A}^{3 / 2}}$$

is necessary to obtain dimensionless measures. The interpretations of the measures CS1′ to CS5′ are the same as in the previous section,

In step 1308, a new set of symmetry measures is calculated which is based on the distance between the regions and the centroid of the lesion G0. Instead of computing the N(N−1)/2 distances between pairs of region centroids (for example point G4 and G5) the N distances between region centroids and the overall lesion centroid are calculated. In the example of FIG. 14A the distances to be measured are G0 to G1; G0 to G2; G0 to G3; G0 to G4; and G0 to G5.

In the case of a symmetric lesion one would expect all the region centroids to be near one another.

From the set of region/centroid distances, the following statistics are calculated:

the minimum distance between any region and the centroid;

the maximum distance between any region and the centroid;

the sum of the distances between all regions and the centroid;

the average of the distances between the regions and the centroid; and

the weighted sum of distances, with weight 1/j used for the j^{th} largest distance.

For the regions/centroid distances, the area-based scaling used is l/logA.

Categorical Segmentation

The distance measures described with reference to FIG. 13A may be applied to a category image segmented according to any chosen measure. Three preferred ways of segmenting the image are to segment by absolute colour, by relative colours, or by 1-D histogram segmentation. Texture-based segmentation may also be used.

Segmentation Based on Absolute Colour

The segmentation of the lesion based on absolute colour is illustrated in FIG. 13B. In step 1310 a set of absolute colour classes is defined. Because the categorical symmetry measures work best without too many classes, the colour classes defined with reference to FIG. 6A are preferably combined to create eleven classes which are more readily identifiable to human interpreters. The eleven absolute colour classes are:

| Combined colour class | Input colour classes |
|---|---|
| Black | Black |
| Grey | Grey |
| BWV | BWV |
| Blue | Blue1 and Blue2 |
| White | White |
| Dark brown | Dark brown |
| Brown | Brown |
| Tan | Tan |
| Skin | Skin |
| Red | Pink2 and Red2 and Red1 |
| Pink | Pink1 |

In step 1312 the lesion image is classified into regions based on the combined colour classes. Next, in step 1314, the categorical symmetry measures based on inter-regional distances are calculated, as described more fully with reference to FIGS. 13A and 14A. In step 1316 the categorical symmetry measures based on region to centroid distances are calculated, as described more fully with reference to FIGS. 13A and 14A.

Segmentation Based on One-Dimensional Histograms

A further procedure for classifying the lesion image based on a one-dimensional histogram segmentation is shown in FIG. 13C. The procedure is preferably based on the histograms of each of the Red, Green and Blue bands. In one arrangement the input data is subjected to a non-linear stretch to emphasise dark features.

In step 1320, a cumulative histogram cumH is formed from the lesion pixels in colour band cb, where cb indicates the Red, Green or Blue colour band. An example of such a cumulative histogram 1424 is shown in FIG. 14B, in which the x-axis 1420 represents the range of pixel values from 0 to 255. The y-axis 1422 (expressed as a percentage) shows the cumulative number of pixels.

In step 1322 a lower threshold (iLO) and upper threshold (iHI) are defined for the cumulative histogram. In the example of FIG. 14B the lower threshold 1428 is set at the 25% value, ie. 25% of the pixels in the image have a value, in colour baud cb, of less than or equal to iLO, The upper threshold 1426 of the cumulative histogram 1424 is set at 75%. In general, the lower threshold is set at a value percentile, and the upper threshold is set to (100-percentile).

In step 1324, the lesion image in colour band cb is classified as a labelled image, histClas_{cb}. The region outside the wanted region mask is labelled “0” and the remaining regions within the mask are labelled as follows:

| | |
|---|---|
| HistClass_{cb} = 1 | if i < iLO; |
| HistClass_{cb} = 2 | if i ≧ iLO and i < iHI; |
| HistClass_{cb} = 3 | if i ≧ iHI. |

The wanted region mask is the lesion boundary mask with the unwanted hairs and bubbles masked out.

Once the lesion has been classified, the inter-region statistics are calculated in step 1326 and the region/centroid statistics are calculated in step 1328. The procedures of steps 1326 and 1328 are described in more detail above with reference to FIG. 13A and FIG. 14A Steps 1320 to 1328 are preferably performed for each of the colour bands Red, Green and Blue.

Segmentation Using Relative Colours

The aim of the relative colour segmentation is to divide the colour space for a lesion into the natural colour clusters for that lesion in a manner analogous to a human interpreter's definition of colour clusters. This contrasts with the absolute colour classification of FIG. 13B which uses fixed colour cluster definitions for all lesions. In the case of relative colour segmentation the colour clusters are calculated on a per-lesion basis.

In step 1340 of FIG. 13D, the lesion image is retrieved from memory and unwanted areas such as hair and bubbles are masked out. In a preferred arrangement, the unwanted regions of the image are found by dilating a hair and bubble mask (using a 7*7 square). The wanted part of the lesion is the lesion boundary mask with the unwanted regions removed. In one arrangement the input data is subjected to a non-linear stretch to emphasise dark features.

The retrieved lesion image is described in RGB colour space. This means that each pixel in the image is described by three colour coordinates. In step 1342 the colour space is transformed from RGB into a new colour space defined by variables relPC1 and relPC2. The conversion from (R,G,B) to (relPC1, relPC2) is done using a predetermined principal component transform. The predetermined transform is derived by performing a principal components (PC) analysis of a training set of lesion image data obtained from a wide range of images. The transform thus obtained maps the image data into a new space in which most of the variance is contained in the first PC band.

FIG. 14C shows an example of a lesion image 1430 expressed in terms of the relPC1 variable. The image 1430 shows a lesion 1432 and surrounding skin 1433. The image 1430 still includes bubble areas, for example area 1436, and hairs, for example hair 1434.

FIG. 14D shows the same lesion as FIG. 14C, but expressed in terms of variable relPC2. Lesion 1442 is the same as lesion 1432, bubble area 1446 corresponds to bubble area 1436 and the hair 1444 corresponds to the hair 1434. It may be seen that image 1440 exhibits less variance than image 1430.

Next, in step 1434, a bivarate histogram mres of image values is constructed in the transformed space. The method of constructing the histogram will be described in more detail with reference to FIG. 13E. An example of a bivarate histogram mres is shown in FIG. 14E. The x-axis of the histogram 1460 maps variation in relPC1 and the y-axis of histogram 1460 maps variation in relPC2.

In step 1346 a set of labelled seeds, bseeds4, is derived from the peaks of the histogram mres. The method of identifying the labelled seeds is described in more detail with reference to FIG. 13P.

In step 1348 the entire histogram space is divided into multiple regions by performing a watershed transformation of the histogram mres about the seeds bseeds4. An example of the result of this process is shown in FIG. 14J in which the histogram space defined by relPC1 and relPC2 has been segmented into four regions 1470a-d. Each of the regions 1470a-d corresponds to a cluster of pixels of similar colour in the original image space, whether defined in terms of relPC1 and relPC2 or R, G and B.

In step 1350 the populated part of the segmented colour space is found by multiplying segres by a mask of the non-zero portions of the histogram mres. The result of this process is denoted segbvh. An example is shown in FIG. 14K, in which the segmentation of FIG. 14J has been combined with the histogram 1460 to yield the four regions 1480a-d.

In step 1352 the lesion image, expressed in terms of variables relPC1 and relPC2, is segmented into regions using the segmented histogram segbvh. An example is shown in FIG. 14L in which a lesion image 1490 has been segmented into four types of region based on the four groups 1480a, 1480b, 1480c and 1480d. For example, region 1492a corresponds to group 1480a of the bivariate histogram segbvh. Similarly, the region 1492b corresponds to the group 1480b of the histogram segbvh and regions 1492c and 1492d correspond to regions 1480c and 1480d respectively.

Once the lesion has been classified, the inter-region statistics are calculated in step 1354 and the region/centroid statistics are calculated in step 1356. The procedures of steps 1354 and 1356 are described in more detail above with reference to FIGS. 13A and 14A.

The formation of the bivariate histogram mres (step 1344 of FIG. 13D) is preferably carried out using the process of FIG. 13E. In step 1360 the size of the histogram is set to rowsize by rowsize rather than the usual bivariate histogram size of 256*256. The parameter row size is derived from the size of the lesion mask using the following equation:

row size=\sqrt{s} 13

where s is the number of pixels in the wanted region mask.

In step 1362 a histogram is constructed in which jitter has been added to the histogram entries to produce a slight smudging of the histogram. If a histogram entry without jitter is defined by the data pair (x,y), the corresponding histogram entry with jitter is positioned at (xjit,yjit). The new entries with jitter are defined by the following equations:

xjit=rowsize*((x−min PC1+1)/(max PC1−min PC1+2))+2.0*random−1.0

yjit=rowsize*((y−min PC2+1)/(max PC2−min PC2+2))+2.0*random−1.0

where minPC1 and maxPC1 are the minimum and maximum values of relPC1 respectively and minPC2 and maxPC2 are the minimum and maximum values of relPC2, and random is a pseudo-random number in the range 0-1.

In step 1364 the histogram is smoothed with a mean filter of radius smoothradius to make a continuous curve.

In step 1366 the dynamic range of the bivariate histogram is stretched such that the histogram has a minimum value of 0 and a maximum value of 255. The output of step 1366 is the bivariate histogram mres.

FIG. 13F shows in more detail the process by which seeds bseeds4 are derived from the peaks of the bivariate histogram mres (ie. step 1346 of FIG. 13D).

In step 1370 the peaks of the histogram mres which have a height greater than a parameter dynamic are removed to obtain the modified histogram noses. This is performed by a morphological reconstruction by dilation of the histogram (mres-dynamic) under mres thereby effectively taking the marker or reference image (mres-dynamic) and iteratively performing geodesic dilations on this image under the mask image mres until idempotence is achieved. In step 1372 the difference between the histogram and the histogram shorn of its peaks (ie. mres-rmres) is thresholded to find those peaks which exceed a specified threshold. The peaks thus located are candidate peak seeds. This is illustrated in FIG. 14F which shows candidate peak seeds 1450a-d derived from bivariate histogram 1460.

In step 1374 those candidate seeds which are sufficiently close together are merged by doing a morphological closing of size closedim*closedim. This process is illustrated in FIG. 14G which shows a set of merged seeds 1452a-d which correspond to the original candidate peak seeds 1450a-d, In this particular example the merging step has not produced any merging apparent within the resolution of the Figures. The parameter closedim is dependent on the parameter rowsize such that the smaller closing is used on histograms of small size and a larger closing is used on histograms of large size.

In step 1376 each connected seed object is labelled to produce a set of labelled objects bseeds3. This is illustrated in FIG. 14H in which object 1454a is a first labelled object, object 1454b is a second labelled object, object 1454c is a third labelled object and object 1454d is a fourth labelled object.

In step 1376 the labels of each merged peak object are transferred on to the original set of candidate peaks seeds bseeds to produce a set of labelled objects bseed4, where bseeds4=bseeds3*bseeds. This is illustrated in FIG. 141 in which the labels associated with objects 1454a-d have been transferred to candidate seeds 1450a-d to produce the four labelled objects 1456a-d.

The objects bseeds4 are then used to segment the histogram space into multiple regions as described in step 1348 of FIG. 13D.

The foregoing methods quantify features of a lesion relating to the colour, shape and texture of the lesion. Measures of the categorical symmetry of the lesion are also obtained. The resulting measures may be assessed by a clinician during diagnosis of the lesion. Additionally, or as an alternative, the resulting measures may be supplied to a classifier for automatic assessment.

### INDUSTRIAL APPLICABILITY

It is apparent from the above that the arrangements described are applicable to the assisted diagnosis of dermatological anomalies.

The foregoing describes only some embodiments of the present disclosure, and modifications and/or changes can be made thereto without departing from the scope and spirit of the disclosure, the embodiments being illustrative and not restrictive.

AUSTRALIA ONLY In the context of this specification, the word “comprising” means “including principally but not necessarily solely” or “having” or “including” and not “consisting only of”. Variations of the word comprising, such as “comprise” and “comprises” have corresponding meanings

1. A method of quantifying features of a skin lesion for use in diagnosis of the skin lesion, the method comprising the steps of:
obtaining an image of an area of skin including the lesion;
segmenting the image into a lesion area and a non-lesion area;
quantifying at least one colour feature of the lesion area;
quantifying at least one shape feature of the lesion area;
calculating at least one symmetry measure descriptive of the distribution of classified regions within the lesion area; and
storing the at least one colour feature, the at least one shape feature and the at least one symmetry measure for use in diagnosis of the skin lesion.

2. A method of quantifying features of a skin lesion for use in diagnosis of the skin lesion, the method comprising the steps of
obtaining a calibrated colour image of an area of skin including the lesion, the image comprising a set of visual elements;
segmenting the image into a lesion area and a non-lesion area;
allocating each visual element in the lesion area to a corresponding one of a predefined set of colour classes;
calculating at least one statistic describing the distribution of the allocated visual elements; and
storing the at least one statistic for further processing as a feature of the lesion.

3. A method as claimed in claim 2 in which the at least one statistic is selected from the group consisting of:
a total number of visual elements allocated to any one of the set of colour classes;
a sum of all visual elements allocated to the set of colour classes;
said total number divided by said sum;
a combined total of visual elements allocated to a predefined combination of colour classes; and
said combined total divided by said sum.

4. A method as claimed in claim 2 in which said visual elements are charaterised by coordinates in a colour space and said allocating step comprises the sub-steps of:
comparing, for each visual element, the coordinates with a predefined lookup table; and
allocating the visual element based on said comparison.

5. A method as claimed in claim 4 in which the predefined lookup table is created by the steps of:
collecting a training set of lesion data manually segmented into labelled colour classes;
generating surfaces in the colour space which best segment the colour space according to the labelled colour classes; and
preparing the lookup table from the surfaces.

6. A method as claimed in claim 5 in which said generating step uses a canonic variate analysis.

7. A method as claimed in any one of claims 2 to 6 in which the predefined set of colour classes is selected from the group consisting of Black, Grey, Blue-White-Veil (BWV), Blue1, Blue2, Darkbrown, Brown, Tan, Pink1, Pink2, Red1, Red2, Skin and White.

8. A method as claimed in claim 7 in which the predefined combination of colour classes is selected from the group consisting of:
a Reds class formed from Pink2 plus Red2 plus Red1;
a Haemangioma class formed from Pink2 plus Red2 plus Red1 plus Pink1;
a BWVBlues class formed from Grey plus Blue-White-Veil;
a Blues class formed from Grey plus Blue-White-Veil plus Blue1 plus Blue2;
a Blue-Whites class formed from Grey plus Blue-White-Veil plus Blue1 plus Blue2 plus White;
a TanSkin class formed from Tan plus Skin; and
a RedBlues class formed from the Haemangioma class plus the Blues class.

9. A method as claimed in claim 2 in which the predefined set of colour classes comprises Blue-White-Veil and Non-Blue-White-Veil.

10. A method as claimed in claim 9 in which the at least one statistic is selected from the group consisting of:
a total number of visual elements allocated to Blue-White-Veil; and
a flag that is set to TRUE if at least a predefined number of spatially contiguous visual elements are allocated to Blue-White-Veil.

11. A method as claimed in claim 9 or 10 in which said visual elements are characterised by coordinates in a colour space and said allocating step comprises the sub-steps of:
comparing, for each visual element, the coordinates with a BWV lookup table;
allocating the visual element according to said comparison.

12. A method as claimed in claim 11 in which the BWV lookup table is created by the steps of:
capturing a manually-assembled training set of BWV data,
constructing a histogram of the training set in the colour space;
forming the BWV lookup table to define a 95% confidence region of the histogram.

13. A method of quantifying features of a skin lesion for use in diagnosis of the skin lesion, the method comprising the steps of:
obtaining a calibrated image of an area of skin including the lesion, the image comprising a set of visual elements;
segmenting the image into a lesion area and a non-lesion area;
assigning a constant value to each visual element in the lesion area to form a binary lesion image;
isolating one or more notches in the binary lesion image;
calculating at least one statistic describing the one or more notches; and
storing the at least one statistic for further processing as a feature of the lesion.

14. A method as claimed in claim 13 wherein the isolating step comprises the substeps of
performing a morphological closing of the binary lesion image to form a closed lesion image;
subtracting the binary lesion image from the closed lesion image to produce one or more difference regions;
performing a morphological opening of the one or more difference regions to produce the one or more notches.

15. A method as claimed in claim 13 or 14 wherein the at least one statistic is selected from the group consisting of:
a total number of notches;
a notch depth measured as a greatest geodesic distance of a notch from an edge of the notch that coincides with an edge of the closed lesion image;
a mean notch depth;
a greatest notch depth;
a mean notch width;
a notch eccentricity measured as the ratio of a notch width to a notch depth;
a mean notch eccentricity,
a largest notch eccentricity; and
a largest notch area.

16. A method of quantifying features of a skin lesion for use in diagnosis of the skin lesion, the method comprising the steps of:
obtaining a calibrated image of an area of skin including the lesion, the image comprising a set of visual elements wherein each visual element has a value;
calculating a lesion boundary that segments the image into a lesion area and a non-lesion area;
calculating an average of the value of each visual element lying on the lesion boundary to form a boundary average;
generating a plurality of outer contours in the non-lesion area such that each outer contour follows the lesion boundary at a respective predetermined distance;
generating a plurality of inner contours in the lesion area such that each inner contour follows the lesion boundary at a respective predetermined distance;
for each one of the inner and outer contours, calculating an average of the value of each visual element lying on the contour to form a contour average;
plotting the contour averages and boundary average against distance to form an edge profile;
calculating an edge abruptness measure from the edge profile; and
storing the edge abruptness measure for further processing as a feature of the lesion.

17. A method as claimed in claim 16 wherein the step of calculating the edge abruptness measure comprises the substeps of:
normalising the edge profile;
finding a mid-point of the normalised edge profile;
defining a left shoulder region lying within a predefined distance range of the mid-point;
defining a right shoulder region lying within the predefined distance range;
calculating a right area from the right shoulder region and a left area from the left shoulder area;
calculating the edge abruptness measure as the sum of the left area and the right area.

18. A method of quantifying features of a skin lesion for use in diagnosis of the skin lesion, the method comprising the steps of:
obtaining a calibrated colour image of an area of skin including the lesion, the image comprising a set of visual elements;
dividing the image into a lesion area and a non-lesion area;
segmenting the lesion area into one or more classes, each class comprising at least one sub-region, such that all visual elements in a class satisfy a predefined criterion;
calculating at least one statistic describing the spatial distribution of the classes;
storing the at least one statistic for further processing as a feature of the lesion.

19. A method as claimed in claim 18 wherein the at least one statistic is selected from the group consisting of;
a centre of gravity of a class,
a first distance between the centre of gravity of one of the classes and the centre of gravity of another one of the classes;
a second distance between the centre of gravity of one of the classes and the centre of gravity of the lesion area;
a third distance between the centre of gravity of a first class having a first area and the centre of gravity of a second class having a second area that is smaller than the first area, wherein the third distance is weighted by the second area;
a maximum distance;
a minimum distance;
an average distance; and
a sum of distances.

20. A method as claimed in claim 18 or 19 wherein the segmenting step comprises the substeps of:
associating each visual element in the lesion with a corresponding one of a predefined set of colour classes;
segmenting the lesion area into the one or more classes wherein said predefined criterion comprises all visual elements in a class being associated with a common one of the colour classes.

21. A method as claimed in claim 18 or 19 wherein each visual element has a descriptive parameter and the segmenting step comprises the fit her substeps of:
constructing a cumulative histogram of all visual elements in the lesion area according to the descriptive parameter;
dividing the cumulative histogram into a plurality of sectors; and
segmenting the lesion area into the one or more classes wherein said predefined criterion comprises all visual elements in a class being associated with a common one of the plurality of sectors.

22. A method as, claimed in claim 21 wherein the colour image is defined ill RGB space and the descriptive parameter is selected from the group consisting of an R-coordinate, a G-coordinate and a B-coordinate.

23. A method as claimed in claim 21 or 22 wherein the plurality of sectors comprises a first sector lying below a low threshold, a second sector lying above a high threshold and a third sector lying between the low threshold and the high threshold.

24. A method as claimed in claim 18 or 19 wherein the visual elements are defined in a first colour space and the segmenting step comprises the substeps of:
transforming the first colour space to a two-dimensional colour space using a predetermined transform;
forming a bivariate histogram of the visual elements in the lesion area, the visual elements being defined in the two-dimensional colour space;
identifying one or more seed regions based on the peaks of the bivariate histogram;
dividing a populated part of the two-dimensional colour space into a plurality of category regions derived from the seed regions; and
segmenting the lesion area into the one or more classes wherein said predefined criterion comprises all visual elements in a class being associated with a common one of the category regions.

25. A method as claimed in claim 24 wherein a method of obtaining the predetermined transform comprises the steps of:
gathering lesion training data defined in the first colour space;
performing a principal component (PC) analysis of the lesion training data to find a first PC axis and a second PC axis;
defining the two-dimensional colour space in terms of the first PC axis and the second PC axis, and
calculating a linear transform that maps the first colour space to the two-dimensional colour space, said linear transform being said predetermined transform.

26. A method as claimed in claim 24 or 25 wherein the step of forming the bivariate histogram comprises the substeps of:
setting a size of the bivariate histogram according to a total number of visual elements in the lesion area;
adding jitter to the bivariate histogram; and
stretching the dynamic range of the bivariate histogram.

27. A method as claimed in any one of claims 24 to 26 wherein the step of identifying the one or more seed regions comprises the substeps of:
shearing the peaks from the bivariate histogram to form a sheared histogram;
thresholding a difference between the bivariate histogram and the sheared histogram to form one or more candidate seeds;
merging candidate seeds that are close together to form one or more merged seeds;
assigning a label each one of the merged seeds; and
transferring the labels to the candidate seeds to form the seed regions.

28. A method of quantifying features of a skin lesion for use in diagnosis of the skin lesion, the method comprising the steps of:
obtaining a calibrated colour image of an area of skin including the lesion, the image comprising a set of visual elements described by coordinates in a colour space;
segmenting the image into a lesion area and a non-lesion area;
comparing, for each visual element, the coordinates with a predefined lookup table;
allocating the visual element to a corresponding one of a predefined set of colours based on said comparison;
calculating at least one statistic describing the distribution of the allocated visual elements; and
storing the at least one statistic for further processing as a feature of the lesion.

29. A method as claimed in claim 28 in which the predefined lookup table is created by the steps of:
collecting a training set of lesion data manually segmented into labelled colour classes;
generating surfaces in the colour space which best segment the colour space according to the labelled colour classes; and
preparing the lookup table from the surfaces.

30. A method as claimed in claim 28 in which the lookup table is created by the steps of:
manually assembling a training set of a predefined melanoma colour;
constructing a histogram of the training set in the colour space;
forming the lookup table to define a 95% confidence region of the histogram.

31. A method of quantifying features of a skin lesion for use in diagnosis of the skin lesion, the method comprising the steps of:
obtaining a calibrated image of an area of skin including the lesion, the image comprising a set of visual elements;
segmenting the image into a lesion area and a non-lesion area;
assigning a constant value to each visual element in the lesion area to form a binary lesion image;
performing a morphological closing of the binary lesion image to form a closed lesion image;
subtracting the binary lesion image from the closed lesion image to produce one or more difference regions;
performing a morphological opening of the one or more difference regions to produce one or more notches;
calculating at least one statistic describing the one or more notches; and
storing the at least one statistic for further processing as a feature of the lesion.

32. A method of quantifying features of a skin lesion for use in diagnosis of the skin lesion, the method comprising the steps of:
obtaining a calibrated image of an area of skin including the lesion, the image comprising a set of visual elements wherein each visual element has a value;
calculating a lesion boundary that segments the image into a lesion area and a non-lesion area;
calculating an average of the value of each visual element lying on the lesion boundary to form a boundary average;
generating a plurality of outer contours such that each outer contour follows the lesion boundary at a predetermined distance;
generating a plurality of inner contours such that each inner contour follows the lesion boundary at a predetermined distance;
for each one of the inner and outer contours, calculating an average of the value of each visual element lying on the contour to form a contour average;
plotting the contour averages and boundary average against distance to form an edge profile;
normalising the edge profile;
finding a mid-point of the normalised edge profile;
defining a left shoulder region lying within a predefined distance range of the mid-point;
defining a right shoulder region lying within the predefined distance range;
calculating a right area from the right shoulder region and a left area from the left shoulder area;
calculating an edge abruptness measure as the sum of the left area and the right area; and
storing the edge abruptness measure for further processing as a feature of the lesion.

33. A method of quantifying features of a skin lesion for use in diagnosis of the skin lesion, the method comprising the steps of:
obtaining a calibrated colour image of an area of skin including the lesion, the image comprising a set of visual elements;
dividing the image into a lesion area and a non-lesion area;
associating each visual element in the lesion with a corresponding one of a predefined set of colour classes;
segmenting the lesion area into one or more classes, each class having at least one-sub-region, such that all visual elements in a class are associated with a common one of the colour classes;
calculating at least one statistic describing the spatial distribution of the classes; and
storing the at least one statistic for further processing as a feature of the lesion.

34. A method of quantifying features of a skin lesion for use in diagnosis of the skin lesion, the method comprising the steps of:
obtaining a calibrated colour image of an area of skin including the lesion, the image comprising a set of visual elements having a descriptive parameter;
dividing the image into a lesion area and a non-lesion area;
constructing a cumulative histogram of all visual elements in the lesion area according to the descriptive parameter;
dividing the cumulative histogram into a plurality of sectors;
segmenting the lesion area into one or more classes, each class having at least one sub-region, such that all visual elements in a class are associated with a common one of the plurality of sectors;
calculating at least one statistic describing the spatial distribution of the classes; and
storing the at least one statistic for further processing as a feature of the lesion.

35. A method of quantifying features of a skin lesion for use in diagnosis of the skin lesion, the method comprising the steps of:
obtaining a calibrated colour image of an area of skin including the lesion, the image comprising a set of visual elements defined in a first colour space;
dividing the image into a lesion area and a non-lesion area;
transforming the first colour space to a two-dimensional colour space using a predetermined transform
forming a bivariate histogram of the visual elements in the lesion area;
identifying one or more seed regions based on the peaks of the bivariate histogram;
dividing a populated part of the two-dimensional colour space into a plurality of category regions derived from the seed regions;
segmenting the lesion area into one or more classes, each class comprising at least one sub-region, such that all visual elements in a class are associated with a common one of the category regions;
calculating at least one statistic describing the spatial distribution of the classes; and
storing the at least one statistic for further processing as a feature of the lesion.

36. A method of quantifying features of a skin lesion for use in the diagnosis of the skin lesion, the method comprising the steps of:
obtaining a calibrated colour image of the lesion;
calculating at least one statistic describing a lesion feature selected from the group consisting of a variance of the lesion; a network measure; a number of dark blobs; and a number of spots in a border region of the lesion.

37. A method of quantifying features of a skin lesion for use in the diagnosis of the skin lesion, the method comprising the steps of:
obtaining a calibrated colour image of the lesion;
obtaining a binary mask of the lesion;
fitting a first ellipse to the binary mask;
fitting a second ellipse to the colour image;
calculating at least one statistic relating to the first ellipse and the second ellipse.

38. A method of quantifying features of a skin lesion for use in the diagnosis of the skin lesion, the method comprising the steps of,
obtaining a calibrated colour image of the lesion;
finding an axis of symmetry of the lesion image;
flipping the lesion image about the axis of symmetry to form a flipped image;
calculating at least one statistic relating to a difference between the lesion image and the flipped image.

39. A method of quantifying features of a skin lesion for use in the diagnosis of the skin lesion, the method comprising the steps of:
obtaining a calibrated colour image of the lesion;
finding a centroid of the lesion image;
dividing the lesion into a plurality of radial segments centred on the centroid;
for each radial segment, calculating a radial array; and
calculating at least one statistic relating to a mean and a variance of the radial arrays.

40. A method of quantifying features of a skin lesion for use in the diagnosis of the skin lesion, the method comprising the steps of:
obtaining a calibrated colour image of the lesion;
finding a centroid of the lesion image;
dividing the lesion into a plurality of radial segments centred on the centroid;
for each radial segment, calculating a radial array;
for each radial array, calculating a Fourier transform to form transform arrays;
finding a correlation between one of said transform arrays and at least one other of said transform arrays;
calculating at least one statistic relating to said transform arrays and said correlation.

41. Apparatus for quantifying features of a skin lesion for use in diagnosis of the skin lesion, the apparatus comprising:
image capture means for obtaining a calibrated colour image of an area of skin including the lesion, the image comprising a set of pixels;
border-finding means for segmenting the image into a lesion area and a non-lesion area;
sorting means for allocating each pixel in the lesion area to a corresponding one of a predefined set of colour classes;
analysis means for calculating at least one statistic describing the distribution of the allocated pixels; and
memory means for storing the at least one statistic for further processing as a feature of the lesion.

42. Apparatus for quantifying features of a skin lesion for use in diagnosis of the skin lesion, the apparatus comprising:
image capture means for obtaining an image of an area of skin including the lesion;
segmentation means for dividing the area into a lesion area and a non-lesion area and defining a binary image of the lesion area;
identification means for isolating one or more notches in the binary image;
analysis means for calculating at least one statistic describing the one or more notches; and
memory means for storing the at least one statistic for further processing as a feature of the lesion.

43. Apparatus for quantifying features of a skin lesion for use in diagnosis of the skin lesion, the apparatus comprising:
image capture means for obtaining a calibrated image of an area of skin including the lesion, the image comprising a set of pixels wherein each pixel has a value,
boundary-detection means for calculating a lesion boundary that segments the image into a lesion area and a non-lesion area;
means for calculating an average of the value of each pixel lying on the lesion boundary to form a boundary average;
distance-transform means for generating a plurality of outer contours in the non-lesion area such that each outer contour follows the lesion boundary at a respective predetermined distance and for generating a plurality of inner contours in the lesion area such that each inner contour follows the lesion boundary at a respective predetermined distance;
means for calculating, for each one of the inner and outer contours, an average of the value of each visual element lying on the contour to form a contour average;
means for forming an edge profile by plotting the contour averages and boundary average against distance;
means for calculating an edge abruptness measure from the edge profile; and
memory means for storing the edge abruptness measure for further processing as a feature of the lesion.

44. Apparatus for quantifying features of a skin lesion for use in diagnosis of the skin lesion, the apparatus comprising:
image capture means for obtaining a calibrated colour image of an area of skin including the lesion, the image comprising a set of pixels;
means for dividing the image into a lesion area and a non-lesion area;
means for segmenting the lesion area into one or more classes, each class comprising at least one sub-region, such that all visual elements in a class satisfy a predefined criterion;
means for calculating at least one statistic describing the spatial distribution of the classes;
memory means for storing the at least one statistic for further processing as a feature of the lesion.

45. A computer readable medium, having a program recorded thereon, where the program is configured to make a computer execute a procedure for quantifying features of a skin lesion, said program comprising:
code for obtaining a calibrated colour image of an area of skin including the lesion, the image comprising a set of visual elements;
code for segmenting the image into a lesion area and a non-lesion area;
code for allocating each visual element in the lesion area to a corresponding one of a predefined set of colour classes;
code for calculating at least one statistic describing the distribution of the allocated visual elements; and
code for storing the at least one statistic for further processing as a feature of the lesion.

46. A computer readable medium, having a program recorded thereon, where the program is configured to make a computer execute a procedure for quantifying features of a skin lesion, said program comprising:
code for obtaining a calibrated image of an area of skin including the lesion, the image comprising a set of visual elements;
code for segmenting the image into a lesion area and a non-lesion area;
code for assigning a constant value to each visual element in the lesion area to form a binary lesion image;
code for isolating one or more notches in the binary lesion image;
code for calculating at least one statistic describing the one or more notches; and
code for storing the at least one statistic for further processing as a feature of the lesion.

47. A computer readable medium having a program recorded thereon, where the program is configured to make a computer execute a procedure for quantifying features of a skin lesion, said program comprising:
code for obtaining a calibrated image of an area of skin including the lesion, the image comprising a set of visual elements wherein each visual element has a value;
code for calculating a lesion boundary that segments the image into a lesion area and a non-lesion area;
code for calculating an average of the value of each visual element lying on the lesion boundary to form a boundary average;
code for generating a plurality of outer contours in the non-lesion area such that each outer contour follows the lesion boundary at a respective predetermined distance;
code for generating a plurality of inner contours in the lesion area such that each inner contour follows the lesion boundary at a respective predetermined distance;
code for calculating, for each one of the inner and outer contours, an average of the value of each visual element lying on the contour to form a contour average;
code for plotting the contour averages and boundary average against distance to form an edge profile;
code for calculating an edge abruptness measure from the edge profile; and
code for storing the edge abruptness measure for further processing as a feature of the lesion.

48. A computer readable medium, having a program recorded thereon, where the program is configured to make a computer execute a procedure for quantifying features of a skin lesion, said program comprising
means for obtaining a calibrated colour image of an area of skin including the lesion, the image comprising a set of visual elements;
means for dividing the image into a lesion area and a non-lesion area;
means for segmenting the lesion area into one or more classes, each class comprising at least one sub-region, such that all visual elements in a class satisfy a predefined criterion;
means for calculating at least one statistic describing the spatial distribution of the classes;
means for storing the at least one statistic for further processing as a feature of the lesion.

49. A method of quantifying features of a skin lesion for use in diagnosis of the skin lesion substantially as described herein with reference to the embodiments as illustrated in the accompanying drawings.

50. Apparatus for quantifying features of a skin lesion for use in the diagnosis of the skin lesion substantially as described herein with reference to the embodiments as illustrated in the accompanying drawings.

51. A computer readable medium substantially as described herein with reference to the embodiments as illustrated in the accompanying drawings.
